(19) United States
(12) Patent Application Publication
(10) Pub. No.: US 2015/0374803 A1
(43) Pub. Date: Dec. 31, 2015

(54) ADENO-ASSOCIATED VIRUS VECTORS AND METHODS OF USE THEREOF

(71) Applicant: The Children's Hospital Of Philadelphia, Philadelphia, PA (US)

(72) Inventor: John H. Wolfe, Blue Bell, PA (US)

(21) Appl. No.: 14/850,292

(22) Filed: Sep. 10, 2015

Related U.S. Application Data
(63) Continuation-in-part of application No. PCT/US2014/025794, filed on Mar. 13, 2014.
(60) Provisional application No. 61/780,423, filed on Mar. 13, 2013.

(51) Int. Cl. A61K 38/47; C12N 9/24; C12N 7/00; C12N 15/86

(52) U.S. Cl. CPC A61K 38/47; C12N 15/86; C12N 9/2402; C12N 7/00; C12N 2750/00043; C12N 2750/00045; C12Y 302/01031; A61K 48/00

(57) ABSTRACT

The present invention provides AAV vectors and methods of use thereof for delivery of transgenes or therapeutic nucleic acids to subjects.

This application is a continuation-in-part of PCT/US2014/025794, filed on Mar. 13, 2014, which claims priority under 35 U.S.C. §119(e) to U.S. Provisional Patent Application No. 61/780,423, filed Mar. 13, 2013. The foregoing application is incorporated by reference herein.

This invention was made with government support under R01NS038690 awarded by the National Institute of Neurological Disorders and Stroke (NINDS) and R01DK063973 awarded by the National Institute of Diabetes and Digestive and Kidney Diseases (NIDDK). The government has certain rights in the invention.

### FIELD OF THE INVENTION

This application relates to the fields of gene therapy and molecular biology. More specifically, this invention provides adeno-associated viral vectors with improved gene transfer to the brain.

### BACKGROUND OF THE INVENTION

Several publications and patent documents are cited throughout the specification in order to describe the state of the art to which this invention pertains. Each of these citations is incorporated herein by reference as though set forth in full.

Adeno-associated virus is a helper-dependent virus (Dependovirus) of the family parvoviridae and requires a helper virus for replication. After infection, the AAV typically enters a latent phase where the AAV genome is site specifically integrated into host chromosomes. The AAV genome is only rescued, replicated, and packaged into infectious viruses again upon an infection with a helper virus. Accordingly, natural infections take place in the context of infection with a helper virus, such as adenovirus or herpes simplex virus.

Not only are AAV vectors nonpathogenic and result in long-term expression of the encoded heterologous gene, but they are also capable of transducing non-dividing cells, which is necessary for treatment of the central nervous system (CNS). Adeno-associated virus (AAV) vectors are scalable, efficient, non-cytopathic gene delivery vehicles used primarily for the treatment of genetic diseases. Indeed, a broad spectrum of animal models of human diseases has been successfully treated by AAV vectors, including diseases of the brain, heart, lung, eye and liver (Mingozzi et al. (2011) Nat. Rev. Genet., 12:341-355). Further, numerous clinical trials with AAV vectors are currently ongoing with positive results in the treatment of a variety of diseases including, for example, Leber's Congenital Amaurosis, hemophilia, congestive heart failure, lipoprotein lipase deficiency, and Parkinson's disease (Maguire et al. (2008) New Eng. J. Med., 358:2240-2248; Bainbridge et al. (2008) New Eng. J. Med., 358:2231-2239; Hauswirth et al. (2008) Human Gene Ther., 19:979-990; Nathwani et al. (2011) New Eng. J. Med., 365:2357-2365; Jessup et al. (2011) Circulation 124:304-313; LeWitt et al. (2011) Lancet Neurol., 10:309-319). Despite the promise of AAV based gene therapy approaches for the treatment of a variety of disorders, improved AAV vectors with specific delivery to target tissues are desired.

### SUMMARY OF THE INVENTION

In accordance with the present invention, compositions and methods for improved delivery of a nucleic acid molecule to the brain, particularly the neurons therein, are provided. In a particular embodiment, the method comprises administering to a subject an AAV vector comprising the nucleic acid molecule of interest, wherein the AAV vector comprises hu.32 or rh.8 capsid proteins or variants thereof. In a particular embodiment, the capsid protein comprises at least 90%, 95%, or more homology/identity with SEQ ID NO: 1 or 3 or is encoded by a nucleic acid molecule having at least 90%, 95%, or more homology/identity with SEQ ID NO: 2 or 4. The AAV may be delivered to the subject intravascularly, e.g., as part of a composition comprising at least one pharmaceutically acceptable carrier.

In accordance with another aspect of the present invention, therapeutic methods for treatment, inhibition, and/or prevention of a disease or disorder, particularly a genetic disease associated with the brain, are provided. In a particular embodiment, the disease or disorder effects more than the brain (e.g., the disease or disorder is a multi-organ disease or disorder (e.g., LSD)). In a particular embodiment, the method comprises administering to a subject an AAV vector comprising a nucleic acid molecule encoding a therapeutic protein or inhibitory nucleic acid molecule, wherein the AAV vector comprises hu.32 or rh.8 capsid proteins or variants thereof. In a particular embodiment, the capsid protein comprises at least 90%, 95%, or more homology/identity with SEQ ID NO: 1 or 3 or is encoded by a nucleic acid molecule having at least 90%, 95% or morehomology/identity with SEQ ID NO: 2 or 4. The AAV may be delivered to the subject intravascularly, e.g., as part of a composition comprising at least one pharmaceutically acceptable carrier and, optionally, at least one other therapeutic agent.

### BRIEF DESCRIPTION OF THE DRAWINGS

FIG. 1A provides an amino acid sequence of hu.32 capsid (SEQ ID NO: 1). FIG. 1B provides a nucleotide sequence of hu.32 capsid (SEQ ID NO: 2). FIG. 1C provides an amino acid sequence of rh.8 capsid (SEQ ID NO: 3). FIG. 1D provides a nucleotide sequence of rh.8 capsid (SEQ ID NO: 4).

FIGS. 2A and 2B provide images of various regions of the mouse brain depicting AAV infection as evidenced by GFP expression.

FIGS. 3A-3D provide images of various regions of the mouse brain depicting AAV infection as evidenced by green fluorescent protein (GFP) expression. FIG. 3A is AAV2/hu32, FIG. 3B is AAV2/rh8, FIG. 3C is AAV2/9, and FIG. 3D is AAV2/hull.

FIG. 4 provides images of various regions of the feline brain depicting AAV infection as evidenced by GFP expression.

FIG. 5A provides images of brain slices from the cortex (ctx), hippocampus (hp), cerebellum (cer), and striatum (str) showing GFP expression indicating AAV infection and NeuN (Fox-3) staining indicating neurons. FIG. 5B provides images of brain slices from the cortex (ctx), hippocampus (hp), and striatum (str) showing GFP expression indicating AAV infection and glial fibrillary acidic protein (GFAP) staining indicating astrocytes. FIG. 5C provides images of brain slices from the cortex (ctx) and striatum (str) showing GFP expression indicating AAV infection and adenomatous polyposis coli (APC) staining indicating oligodendrocytes.

FIG. 6 provides histopathology images of hippocampus, thalamus, and entorhinal cortex brain sections from normal mice, untreated MPS VII mice, and MPS VII mice transduced with AAV.hu32.hGBp.GUSB.

FIG. 7A provides images mouse brain following intravenous delivery of AAV vectors. Intravenous injection of 2.9×10¹² vg of AAV9, hu.11, rh.8 and hu.32 expressing GFP in adult mice results in GFP expression throughout the brain 4 weeks post-injection (n=3 mice for each group). FIG. 7B provides a graph of the amount of transduction quantified by counting the number of GFP-positive objects throughout the brain in sections at distances from Bregma as shown. Scale bar, 500 μm. *P<0.05; **P<0.01; ***P<0.001.

FIG. 8 shows mouse brain transduction following intravenous delivery of AAVhu.32 in different strains of mice. Balb/c, B16 and C3H mice were intravenously injected with 5.8×10¹¹ vg of AAVhu.32-GFP and the transduction in the brain was assessed by counting the number of GFP-positive objects 4 weeks post-injection (n=3 mice for each group). *P<0.05; **P<0.01.

FIG. 9 shows intravenous injection of AAVhu.32 results in predominant neuronal transduction in the CNS of adult mice. The phenotype of the transduced cells in the CNS was verified by dual immunofluorescent staining with antibodies against GFP and a neuronal marker (NeuN) in the striatum, cortex, hippocampus and the spinal cord. Images in the right-hand columns for both GFP and merge are higher magnification pictures of images in the left-hand columns. Scale bars: 100 μm (left columns), 50 μm (right columns).

FIGS. 10A-10C show carotid injection of AAVhu.32 in cats results in broad transduction throughout the brain. Three 6-week-old cats were injected with 2.9×10¹³ vg/kg of AAVhu.32-GFP into the carotid artery and vector transduction was analyzed throughout the brain (FIG. 10A; representative image of various brain sections studied), spinal cord (FIG. 10B), and peripheral organs (FIG. 10C) by immunohistochemistry at 6 weeks post-injection. Negative control brain section with no primary antibody showed no staining Images in the lower panels for spinal cord (FIG. 10B) and peripheral organs (FIG. 10C) are higher magnification pictures of images in the upper panels. Scale bars: 500 μm (FIG. 10A); 600 μm (FIG. 10B, upper panel); 60 μm (FIG. 10B, lower panel); 200 μm (FIG. 10C, upper panels); 50 μm (FIG. 10C, lower panels).

FIG. 11 shows predominant neuronal transduction in the brain by AAVhu.32 following carotid injection of cats. The phenotype of the transduced cells in the CNS was verified by dual immunofluorescent staining with antibodies against GFP and a neuronal marker (NeuN) in the striatum, cortex, hippocampus and the spinal cord. Images in the right-hand columns for both GFP and merge are higher magnification pictures of images in the left-hand columns. Scale bars: 100 μm (left column), 50 μm (right column).

FIGS. 12A-12E show monkey brain transduction following intravascular injection of AAVhu.32. Three monkeys were injected with 1.3×10¹³ vg/kg of AAVhu.32-GFP into the carotid artery and vector transduction was analyzed by immunohistochemistry at 8 weeks post-injection. FIG. 12A provides the locations of the 4 brain sections analyzed in each monkey. The letters indicate the position of the sections shown in FIGS. 12B-12E. FIGS. 12B-12E provide representative brain sections showing vector transduction throughout the brain. Neurons by morphology and glial cells are marked on whole brain images. High magnification images of various structures of the brain from the adjacent sections are shown. Images in the lower panels are higher magnification pictures of transduced cells from the upper panels. Ctx: cortex; CA: caudate nucleus; Pu: putamen; Th: thalamus; Hp: hippocampus; SC: superior colliculus; Mb: midbrain; Cer: cerebellum. Scale bars: 300 μm (upper panels); 60 μm (lower panels).

FIG. 13 is a table of monkey serum chemistry pre- and post-AAVhu.32 intracarotid injection.

### DETAILED DESCRIPTION OF THE INVENTION

Adeno-associated virus (AAV) vectors are among the most promising viral vectors for in vivo gene transfer. The prototype AAV2 vector results inrelatively limited transduction of central nervous system (CNS) cells, and many humans are seropositive for AAV2, thereby limiting its use in clinical applications. However, the cross-packaging of the AAV2 genome with capsid proteins from alternative AAV serotypes has resulted in improved gene transferin a variety of tissues, including the brain (Davidson et al. (2000) Proc. Natl. Acad. Sci., 97:3428-3432; Passini et al. (2003) J. Virol., 77:7034-7040; Burger et al. (2004) Mol. Ther., 10:302-317; Cearley et al. (2006) Mol. Ther., 13:528-537; Taymans et al. (2007) Hum. Gene. Ther., 18:195-206; Cearley et al. (2008) Mol. Ther., 16:1710-1718). Many AAV capsid sequences have been isolated from humans and nonhuman primates by molecular rescue of sequences of endogenous AAVs. The capsid sequences have been phylogenetically characterized into six clades: A through F (Gao et al. (2002) Proc. Natl. Acad. Sci., 99:11854-11859; Gao et al. (2003) Proc. Natl. Acad. Sci., 100:6081-6086; Gao et al. (2004) J. Virol., 78:6381-6388). Certain AAV serotypes have a specific tropism for neurons and are unable to efficiently transduce other cell types within the brain such as astrocytes or oligodendrocytes while other AAV serotypes are able to undergo vector transport along neuronal projections (Davidson et al. (2000) Proc. Natl. Acad. Sci., 97:3428-3432; Burger et al. (2004) Mol. Ther., 10:302-317; Cearley et al. (2006) Mol. Ther., 13:528-537; Kaspar et al. (2003) Science 301:839-842; Passini et al. (2005) Mol. Ther., 11:754-762; Cearley et al. (2007) J. Neurosci., 27:9928-9940; Cearley et al. (2008) Mol. Ther., 16:1710-1718; Foust et al. (2009) Nat. Biotech., 27:59-65).

The instant invention demonstrates that AAV vectors comprising the hu.32 or rh.8, particularly the hu.32, capsid protein mediate AAV vector gene transfer into the brain of mice after intravascular injection. The first two letters of the nomenclature refer to the species of isolation (e.g., hu: human) followed by the number of the isolate from that species. The AAV vector specifically transduces neurons in the brain, especially the cerebral cortex, and is very widespread. The types of cells transduced by the instant AAV vectors along with the amount of distribution within the brain are unique. Further, the instant AAV vector is less efficient in transducing the liver than other AAV serotypes, thereby reducing the untoward immune response to the AAV vector in vivo, a clinical drawback of many AAV vectors. The distribution within the brain makes the AAV vectors of the instant invention excellent vectors for the treatment of a variety of disorders including genetic disorders affecting the brain (including diseases or disorders affecting other parts of the body in addition to the brain) such as lysosomal storage diseases and neurodegenerative diseases (e.g., Alzheimer's disease).

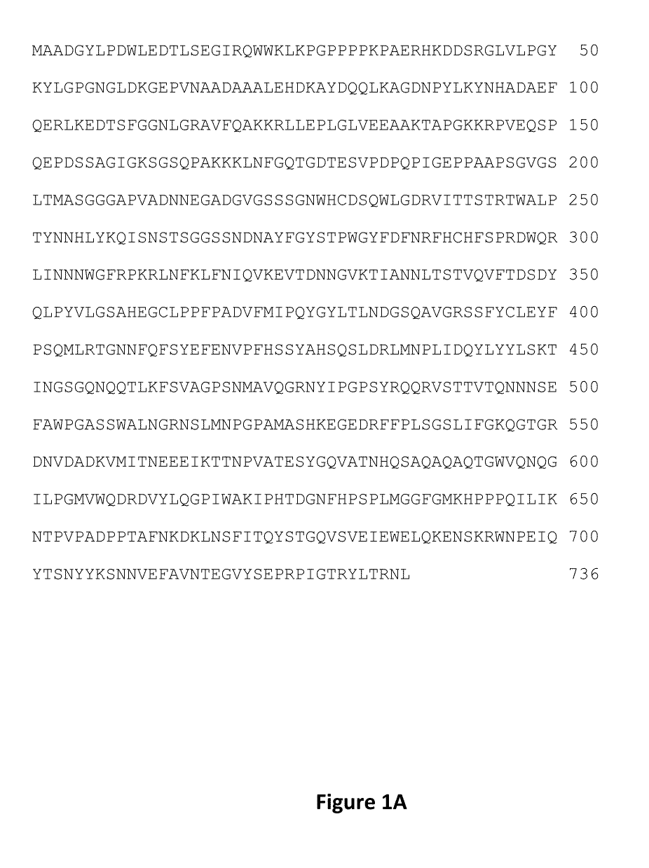

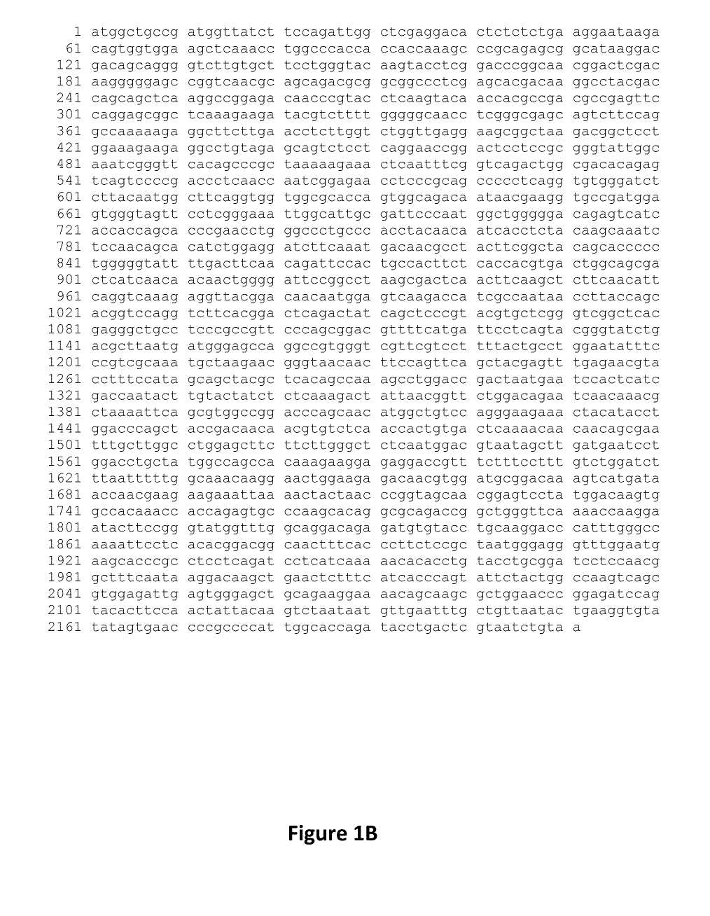

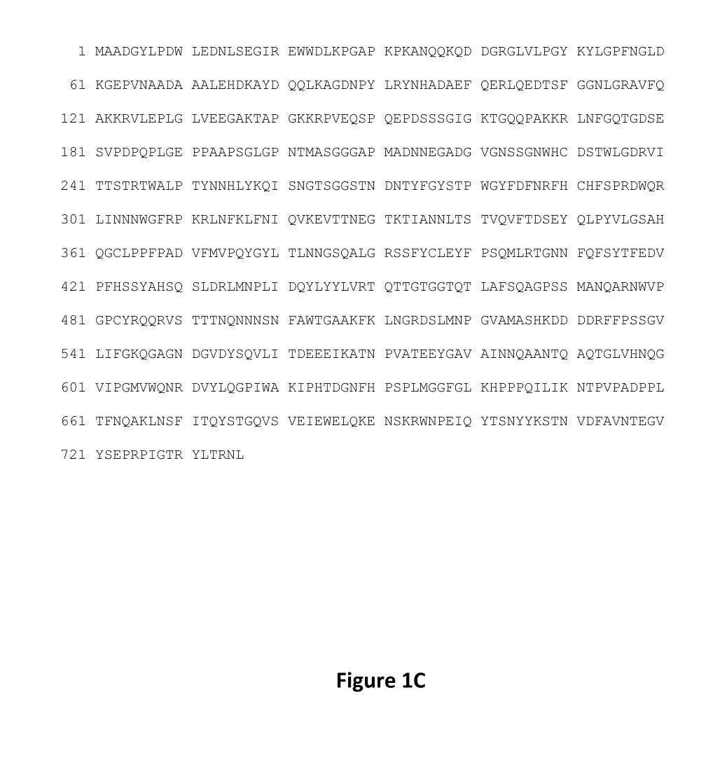

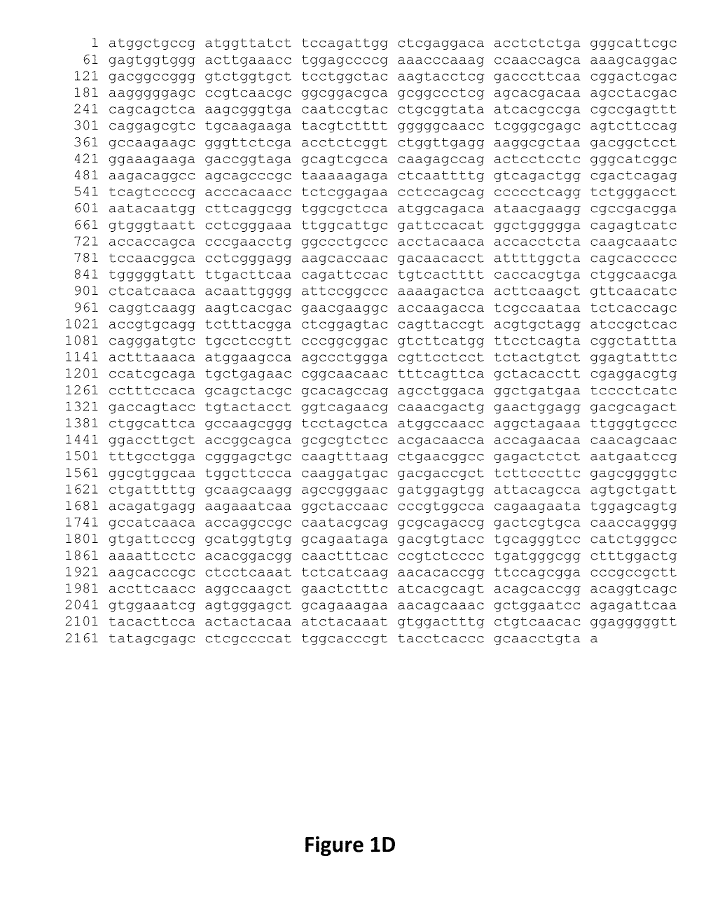

GenBank Accession Nos. AY530597 and AAS99282 provide examples of the amino acid and nucleotide sequences of hu.32 capsid (vp1). GenBank Accession Nos. AAO88183 and AY242997 provide examples of the amino acid and nucleotide sequences of rh.8 capsid (vp1). The AAV capsid is composed of three proteins, vp1, vp2 and vp3, which are alternative splice variants. In other words, vp2 and vp3 are fragments of vp1. FIG. 1A provides SEQ ID NO: 1, which is the wild-type amino acid sequence of hu.32 vp1 capsid. FIG. 1B provides SEQ ID NO: 2, which is the wild-type nucleotide sequence of hu.32 vp1 capsid. FIG. 1C provides SEQ ID NO: 3, which is the wild-type amino acid sequence of rh.8 vp1 capsid. FIG. 1D provides SEQ ID NO: 4, which is the wild-type nucleotide sequence of rh.8 vp1 capsid. The instant invention encompasses variants of the hu.32 and rh.8 capsids. In a particular embodiment, the capsid of the instant invention has an amino acid sequence that is at least 80%, at least 90%, at least 95%, at least 97%, at least 99%, or is 100% identical with SEQ ID NO: 1 or SEQ ID NO: 3. In a particular embodiment, the nucleic acid molecule encoding capsid of the instant invention has a nucleotide sequence that is at least 80%, at least 90%, at least 95%, at least 97%, at least 99%, or is 100% identical with SEQ ID NO: 2 or SEQ ID NO: 4.

The instant invention encompasses methods of delivering a nucleic acid molecule of interest (e.g., heterologous) to cells, particularly in a subject (i.e., in vivo). In a particular embodiment, the method delivers the nucleic acid molecule to neurons (e.g., in the central nervous system including the spinal cord and brain) or the brain, particularly neurons within the brain. In a particular embodiment, the method delivers the nucleic acid molecule to the olfactory bulb, striatum, cortex, hippocampus, hypothalamus, subthalamus, midbrain, brain stem, superior colliculus, inferiotcolliculus, entorhinal cortex, subiculum, and/or cerebellum. The method may comprise contacting the cells with (e.g., by administering to the subject) an AAV vector comprising the hu.32 or rh.8 capsid of the instant invention, wherein the AAV vector comprises the nucleic acid molecule to be delivered. The packaged nucleic acid molecule may encode, for example, a protein of interest (e.g., a therapeutic protein) or an inhibitory nucleic acid molecule (e.g., antisense, siRNA, DsiRNA (Dicer siRNA/Dicer-substrate RNA), shRNA, miRNA (microRNA), etc.). In a particular embodiment, the nucleic acid molecule to be delivered to the subject is a gain-of-function manipulation. The delivery of a nucleic acid molecule of interest in accordance with the instant invention may be used to create a disease model (e.g., a brain disease model) in the subject (e.g., the expression of at least one protein of interest (e.g., a mutant) associated with a disease or disorder). For example, the delivery of a nucleic acid molecule of interest in accordance with the instant invention may be used to create a disease model of a neurodegenerative disease such as Alzheimer's disease (e.g., by expressing at least one gene (e.g., a mutant) associated with Alzheimer's disease (see, e.g., Chin, J. (2011) Methods Mol. Biol., 670:169-89; Mineur et al. (2005) Neural. Plast., 12:299-310; Hall et al. (2012) Brain Res. Bulletin 88:3-12)) or Huntington's disease (e.g., by expressing a mutant huntingtin gene (also known as interesting transcript 15 (IT151) gene) associated with Huntington's disease). The instant invention also encompasses the disease models generated by the methods of the instant invention. The nucleic acid molecule of the instant invention may further comprise appropriate regulatory elements such as promoters or expression operons to express the encoded for protein or inhibitory nucleic acid molecule.

Methods of treating, inhibiting, and/or preventing a disease or disorder in a subject are also encompassed by the instant invention. In a particular embodiment, the method comprises administering to a subject in need thereof an AAV vector comprising the hu.32 or rh.8 capsid of the instant invention, wherein the AAV vector comprises a nucleic acid molecule of interest (e.g., therapeutic nucleic acid molecule) to be delivered. In a particular embodiment, the AAV vector is administered as part of a composition comprising at least one pharmaceutically acceptable carrier. The AAV vectors of the instant invention may be co-administered with any other therapeutic method for the treatment of the disease or disorder. The nucleic acid molecule of the AAV vector may encode a therapeutic protein or a therapeutic inhibitory nucleic acid molecule (e.g., siRNA). The nucleic acid molecule may further comprise appropriate regulatory elements such as promoters or expression operons to express the encoded for protein or inhibitory nucleic acid molecule.

In a particular embodiment, the disease or disorder is a genetic disease or disorder affecting the brain. Examples of the diseases or disorders that may treated include, without limitation: neurological degenerative disorders, Alzheimer's disease, Parkinson's disease, Huntington's disease (HD), stroke, trauma, infections, meningitis, encephalitis, gliomas, cancers (including brain metastasis), multiple system atrophy, progressive supranuclear palsy, Lewy body disease, neuroinflammatory disease, spinal muscular atrophy, amyotrophic lateral sclerosis, neuroAIDS, Creutzfeldt-Jakob disease, Pick's Disease, multi-infarct dementia, frontal lobe degeneration, corticobasal degeneration, HIV-1 associated dementia (HAD), HIV associated neurocognitive disorders (HAND), paralysis, amyotrophic lateral sclerosis (ALS or Lou Gerhig's disease), multiple sclerosis (MS), CNS-associated cardiovascular disease, prion disease, obesity, metabolic disorders, inflammatory disease, metabolic disorders, and lysosomal storage diseases (LSDs; such as, without limitation, Gaucher's disease, Pompe disease, Niemann-Pick, Hunter syndrome (MPS II), mucopolysaccharidosis (MPS) (e.g., mucopolysaccharidosis I (MPS I), mucopolysaccharidosis VII (MPS VII), alpha-mannosidosis etc.), GM2-gangliosidoses, Sanfilippo syndrome (MPS IIIA), Tay-Sachs disease, Sandhoff's disease, Krabbe's disease, metachromatic leukodystrophy, and Fabry disease). In a particular embodiment, the disease or disorder is a lysosomal storage disease.

Gene transfer may be used to provide therapy for a variety of disease states. In general, gene transfer may be used to treat: 1) deficiency states, wherein a protein (e.g., an enzyme) is expressed at abnormally low levels or is defective (e.g., mutated) and has diminished activity, which can be treated by introducing a nucleic acid encoding for the protein (e.g., wild-type protein); and 2) over-expression states, wherein a protein is expressed to abnormally high levels or is defective (e.g., mutated) and has increased or uncontrolled activity, which can be treated by introducing an inhibitory nucleic acid molecule directed against the protein. The use of site-specific integration of nucleic acid sequences to cause mutations or to correct defects is also encompassed by the instant invention.

In a particular embodiment, a therapeutic protein is a peptide or protein that alleviates or reduces symptoms that result from an absence or defect in a protein in a cell or subject. A therapeutic protein may be a peptide or protein that may be used in the treatment of a disease or disorder. Therapeutic proteins include, but are not limited to, enzymes, antibodies, hormones, growth factors, other polypeptides, which administration to cells (e.g., neurons) can effect amelioration and/or cure of a disease, disorder, pathology, and/or the symptoms associated therewith. Neuroactive polypeptides useful in this invention include but are not limited to endocrine factors, growth factors, hypothalamic releasing factors, neurotrophic factors, paracrine factors, neurotransmitter polypeptides, antibodies and antibody fragments which bind to any of the above polypeptides (such as neurotrophic factors, growth factors, and others), antibodies and antibody fragments which bind to the receptors of these polypeptides (such as neurotrophic factor receptors), cytokines, endorphins, enzymes, polypeptide antagonists, agonists for a receptor expressed by a CNS cell, polypeptides involved in lysosomal storage diseases, and the like. In a particular embodiment, the therapeutic protein exerts its effect on the CNS, particularly the brain.

Examples of specific therapeutic proteins include, without limitation, β-glucuronidase (e.g., for the treatment of lysosomal storage disorders), catalase, telomerase, superoxide dismutase (SOD), glutathione peroxidase, glutaminase, cytokines, endorphins (e.g., enkephalin), growth factors (e.g., epidermal growth factor (EGF)), acidic and basic fibroblast growth factor (aFGF and bFGF), insulin-like growth factor I (IGF-I; e.g., Oppenheim, R W (1996) Neuron 17:195-197; Thoenen et al. (1993) Exp. Neurol., 124:47-55; Henderson, C E (1995) Adv. Neurol., 68:235-240), brain-derived neurotrophic factor (BDNF), glial-derived neurotrophic factor (GDNF; e.g., Li et al. (2009) Biochem. Biophys. Res. Comm., 390:947-951), neurotrophin-3 (NT-3), NT-4/5, protease nexin I (PNI; e.g., for the treatment of Alzheimer disease (Houenou et al. (1995) PNAS 92:895-899)), serine protease inhibitor protein (SPI3; e.g., Safaei, R. (1997) Brain Res Dev Brain Res., 100:5-12), platelet derived growth factor (PDGF), vascular growth factor (VGF), nerve growth factor (NGF), insulin-like growth factor-II (IGF-II), tumor necrosis factor-B (TGF-B), survival motor neuron (SMN; e.g., for the treatment of spinal muscular atrophy; Lefebvre et al. (1995) Cell 80:155-165; Roy et al. (1995) Cell 80:167-178), leukemia inhibitory factor (LIF), anti-apoptotic proteins (e.g., BCL-2, PI3 kinase), amyloid beta binders (e.g. antibodies), butyrylcholinesterase or acetylcholinesterase (e.g., Carmona et al. (1999) Drug Metab. Dispos., 28:367-371; Carmona (2005) Eur. J. Pharmacol., 517:186-190), modulators of α-, β-, and/or γ-secretases, vasoactive intestinal peptide, leptin, acid alpha-glucosidase (GAA), acid sphingomyelinase, iduronate-2-sultatase (I2S), α-L-iduronidase (IDU), β-Hexosaminidase A (HexA), β-N-acetylhexosaminidase A Acid β-glucocerebrosidase, N-acetylgalactosamine-4-sulfatase, α-galactosidase A, and neurotransmitters (e.g., Schapira, A H (2003) Neurology 61:S56-63; Ferrari et al. (1990) Adv Exp Med Biol. 265:93-99; Ferrari et al. (1991) J. Neurosci., Res. 30:493-497; Koliatsos et al. (1991) Ann. Neurol. 30:831-840; Dogrukol-Ak et al. (2003) Peptides 24:437-444; Amalfitano et al. (2001) Genet Med. 3:132-138; Simonaro et al. (2002) Am. J. Hum. Genet., 71:1413-1419; Muenzer et al. (2002) Acta Paediatr Suppl. 91:98-99; Wraith et al. (2004) J Pediatr. 144:581-588; Wicklow et al. (2004) Am J Med Genet. 127A:158-166; Grabowski (2004) J Pediatr. 144:S15-19; Auclair et al. (2003) Mol Genet Metab. 78:163-174; Przybylska et al. (2004) J Gene Med. 6:85-92). In a particular embodiment, the therapeutic protein is β-glucuronidase.

While the instant invention is generally described above for the delivery of therapeutic proteins, the AAV of the instant invention may deliver a nucleic acid molecule encoding a detectable protein (e.g., either alone or in combination with a therapeutic protein). Detectable proteins include, without limitation, fluorescent proteins (e.g., GFP), horseradish peroxidase, urease, alkaline phosphatase, glucoamylase, ferritin, dopamine receptor, and β-galactosidase.

Methods of synthesizing AAV vectors are well known in the art (see, e.g., PCT/US04/028817 and Gao et al. (2002) Proc. Natl. Acad. Sci., 99:11854-11859). In a particular embodiment, the method comprises culturing host cells comprising a nucleic acid sequence encoding hu.32 or rh.8 capsid, a nucleic acid encoding rep, and a nucleic acid construct comprising AAV inverted terminal repeats (ITRs) flanking at least the nucleic acid molecule of interest, such that the nucleic acid of interest is packaged in to AAV vectors. In a particular embodiment, a full length AAV genome is used. While a self-complimentary vector (scAAV; such as those typically used with AAV9) may be used in the instant invention, the full coding capacity found in rAAV is about 4.5 kb or larger, whereas scAAV typically have a capacity of about 2.3 kb. Inasmuch as certain proteins of interest (e.g., enzymes) may be encoded by a nucleic acid having a length exceeding the capacity of scAAV, the full length AAV vector would be preferred. The host cell may also provide helper functions (e.g., those supplied by a herpes virus or adenovirus) to package the AAV vectors. The components required of the host cell to package nucleic acid molecules into AAV vectors may be provided in trans or by a stably transduced host cell. The rep gene and/or the AAV ITRs may be from any AAV serotype. For example, the rep gene and/or the AAV ITRs may be from, without limitation, AAV-1, AAV-2, AAV-3, AAV-4, AAV-5, AAV-6, AAV-7, AAV-8, AAV-9, etc. In a particular embodiment, the AAV ITRs are from the AAV2 serotype. The encapsulated nucleic acid molecule may encode more than one protein or polypeptide. When the nucleic acid molecule encodes more than one protein/polypeptide, the encoding regions may be separated by an internal ribozyme entry site (IRES) or nucleic acid sequence encoding a self-cleaving peptide such as a 2A peptide.

The instant invention encompasses methods of treating a disease or disorder in a subject (e.g., a neurological disease or disorder) comprising the administration of a composition comprising the AAV vectors of the instant invention and at least one pharmaceutically acceptable carrier to a subject in need thereof. The term “subject” as used herein refers to human or animal (particularly mammalian) subjects.

The AAV vectors of the invention may be conveniently formulated for administration with any pharmaceutically acceptable carrier. For example, the viral vectors may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the AAV vectors in the chosen medium may be varied and the medium may be chosen based on the desired route of administration of the pharmaceutical preparation. Except insofar as any conventional media or agent is incompatible with the AAV vector to be administered, its use in the pharmaceutical preparation is contemplated.

The dose and dosage regimen of the compositions according to the invention that are suitable for administration to a particular patient may be determined by a physician/veterinarian/medical specialist considering the patient's age, sex, weight, general medical condition, and the specific condition for which the AAV vector is being administered and the severity thereof. The physician/veterinarian/medical specialist may also take into account the route of administration, the pharmaceutical carrier, and the AAV vector's biological activity. Exemplary doses for achieving therapeutic effects are AAV titers of at least about 10⁵, 10⁶, 10⁷, 10⁸, 10⁹, 10¹⁰, 10¹¹, 10¹², 10¹³, 10¹⁴, 10¹⁵, 10¹⁶ transducing units or more, particularly about 10⁸ to 10¹³ transducing units. In particular embodiments of the invention, more than one administration (e.g., two, three, four, or more administrations) may be employed to achieve desired (e.g. therapeutic) levels of gene expression.

Selection of a suitable pharmaceutical preparation will also depend upon the mode of administration chosen. The pharmaceutical preparation comprises the AAV vector preferably dispersed in a medium that is compatible with the site of injection. AAV vectors of the instant invention may be administered by any method such as injection into the blood stream, oral administration, or by subcutaneous, intracranial, intramuscular or intraperitoneal injection. The AAV vector of the invention may be administered by direct injection into an area proximal to or across the blood brain barrier. In a particular embodiment, the composition comprising the AAV vector is administered directly to or to an area proximal to a neuron(s). In a particular embodiment, the composition comprising the AAV vector is administered intravascularly or intravenously. The AAV vectors of the instant invention may be administered into any fluid space of the subject including, without limitation, blood or cerebrospinal fluid (CSF). Pharmaceutical preparations for injection are known in the art. If injection is selected as a method for administering the AAV vectors, steps must be taken to ensure that sufficient amounts of the viral vectors reach their target cells to exert a biological effect.

Pharmaceutical compositions containing an AAV vector the present invention as the active ingredient in intimate admixture with a pharmaceutically acceptable carrier can be prepared according to conventional pharmaceutical techniques. The carrier may take a wide variety of forms depending on the form of preparation desired for administration, e.g., intravascular, direct injection, intracranial, and intramuscular.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

In accordance with the present invention, the appropriate dosage unit for the administration of AAV vectors may be determined by evaluating toxicity, if any, in animal models. Various concentrations of AAV vectors in pharmaceutical preparations may be administered to mice or other animals (e.g., models of the disease to be treated), and the minimal and maximal dosages may be determined based on the beneficial results and side effects observed as a result of the treatment. Appropriate dosage unit may also be determined by assessing the efficacy of the AAV vector treatment in combination with other standard drugs. The dosage units of AAV vector may be determined individually or in combination with each treatment according to the effect detected.

The AAV vectors, reagents, and methods of the present invention can be used to direct a nucleic acid to either dividing or non-dividing cells, and to stably express the nucleic acid therein. The vectors of the present invention can thus be useful in gene therapy for disease states or for experimental modification of cell physiology.

### DEFINITIONS

The singular forms “a,” “an,” and “the” include plural referents unless the context clearly dictates otherwise.

“Gene therapy” is the insertion of nucleic acids (e.g., genes) into an individual's cells and/or tissues to treat a disease or disorder, commonly hereditary or genetic diseases (e.g., wherein a defective mutant allele is replaced or supplemented with a functional one).

The term “treat” as used herein refers to any type of treatment that imparts a benefit to a patient afflicted with a disease, including improvement in the condition of the patient (e.g., in one or more symptoms), delay in the progression of the condition, etc.

A “therapeutically effective amount” of a compound or a pharmaceutical composition refers to an amount effective to prevent, inhibit, treat, or lessen a particular disorder or disease and/or the symptoms associated with it. The treatment of a neurological disease or disorder herein may refer to curing, relieving, inhibiting, and/or preventing the neurological disease or disorder, a symptom(s) of it, or the predisposition towards it.

An “inhibitory nucleic acid molecule” generally refers to small nucleic acid molecules which are capable of modulating expression levels of a target mRNA, (e.g., siRNA, shRNA, miRNA, DsiRNA, antisense oligonucleotides etc.). These molecules may inhibit expression of a target gene involved in mediation of a disease process, thereby preventing or alleviating the disease and/or the symptoms associated with it.

The phrase “small, interfering RNA (siRNA)” refers to a short (typically less than 30 nucleotides long, particularly 12-30 or 20-25 nucleotides in length) double stranded RNA molecule (although the siRNA may be generated by cleavage of longer dsRNA molecules). Typically, the siRNA modulates the expression of a gene to which the siRNA is targeted. siRNAs have homology (e.g., complete complementarity) with the sequence of the cognate mRNA of the targeted gene. Methods of identifying and synthesizing siRNA molecules are known in the art (see, e.g., Ausubel et al., Current Protocols in Molecular Biology, John Wiley and Sons, Inc). Exemplary modifications to siRNA molecules are provided in U.S. Application Publication No. 2005/0032733. Expression vectors for the expression of siRNA molecules preferably employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502-09).

The term “short hairpin RNA” or “shRNA” refers to an siRNA precursor that is a single RNA molecule folded into a hairpin structure comprising an siRNA and a single stranded loop portion of at least one, typically 1-10, nucleotide. shRNA molecules are typically processed into an siRNA within the cell by endonucleases.

As used herein, the term “microRNA” or “miRNA” refers to any type of interfering RNA, including but not limited to, endogenous microRNA (naturally present in the genome) and artificial microRNA. MicroRNA typically have a length in the range of from about 18 to about 30 nucleotides, particularly about 21 to about 25 nucleotides. MicroRNA may be single-stranded RNA molecules. The microRNA may be in the form of pre-miRNA, typically a short stem-loop structure having a length of about 50 to about 90 nucleotides, particularly about 60 to about 80 nucleotides, which are subsequently processed into functional miRNAs.

The term “RNA interference” or “RNAi” refers generally to a sequence-specific or selective process by which a target molecule (e.g., a target gene, protein or RNA) is down-regulated via a double-stranded RNA. The double-stranded RNA structures that typically drive RNAi activity are siRNAs, shRNAs, microRNAs, and other double-stranded structures that can be processed to yield a small RNA species that inhibits expression of a target transcript by RNA interference.

The term “Dicer substrate RNA” or “DsiRNA” refers to oligonucleotides which comprise at least one siRNA molecule and which serve as a substrate for Dicer to release the siRNA molecule, typically 21 nucleotides in length. DsiRNA are double-stranded and comprise RNA or DNA and RNA. Typically, DsiRNA are less than about 100 nucleotides in length, less than about 50 nucleotides in length, less than about 40 nucleotides in length, less than about 35 nucleotides in length, or less than about 30 nucleotides in length. In a particular embodiment, the DsiRNA is 27 nucleotides in length. Examples of DsiRNA are provided in U.S. Patent Application Publication Nos. 2005/0244858; 2005/0277610; 2007/0265220; and 2010/0184841.

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 10 and about 100 nucleotides in length, particularly between about 15 and about 50 nucleotides, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween™ 80, Polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

The term “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art. Inducible promoters, tissue-specific promoters, native promoters, or constitutive or high level promoters may be used. In a particular embodiment, high-level constitutive expression may be desired. Examples of such promoters include, without limitation, the retroviral Rous sarcoma virus (RSV) LTR promoter/enhancer, the cytomegalovirus (CMV) immediate early promoter/enhancer, the SV40 promoter, the dihydrofolate reductase promoter, the cytoplasmic β-actin promoter and the phosphoglycerol kinase (PGK) promoter. In another embodiment, the native promoter for the transgene or nucleic acid sequence of interest is used. The native promoter may be preferred when it is desired that expression of the transgene or the nucleic acid sequence should mimic the native expression. The native promoter may be used when expression of the transgene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression. In a particular embodiment, the tissue-specific promoter is neuron specific. Examples of neuron specific promoters include, without limitation: neuron-specific enolase (NSE) promoter (Andersen et al. (1993) Cell. Mol. Neurobiol., 13:503-15); neurofilament light-chain gene (Piccioli et al. (1991) Proc. Natl. Acad. Sci., 88:5611-5); the neuron-specific vgf gene (Piccioli et al. (1995) Neuron, 15:373-84); and the like.

The term “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

A “vector” is a replicon, such as a plasmid, cosmid, bacmid, phage or virus, to which another genetic sequence or element (either DNA or RNA) may be attached so as to bring about the expression and/or replication of the attached sequence or element.

The term “gene” refers to a nucleic acid comprising an open reading frame encoding a polypeptide, including exon and (optionally) intron sequences. The nucleic acid may also optionally include non-coding sequences such as promoter or enhancer sequences. The term “intron” refers to a DNA sequence present in a given gene that is not translated into protein and is generally found between exons.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “isolated” may refer to protein, nucleic acid, compound, or cell that has been sufficiently separated from the environment with which it would naturally be associated, e.g., so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification.

The term “percent identity” refers to the percentage of sequence identity found in a comparison of two or more nucleic acid sequences. Percent identity can be determined by standard alignment algorithms, for example, the Basic Local Alignment Search Tool (BLAST) described by Altshul et al. (J. Mol. Biol. (1990) 215:403-10) as well as GAP, BESTFIT, FASTA, and TFASTA (available as part of the GCG® Wisconsin Package® (Accelrys Inc., Burlington, Mass.)).

“Polypeptide” and “protein” are sometimes used interchangeably herein and indicate a molecular chain of amino acids. The term polypeptide encompasses peptides, oligopeptides, and proteins. The terms also include post-expression modifications of the polypeptide, for example, glycosylations, acetylations, phosphorylations and the like. In addition, protein fragments, analogs, mutated or variant proteins, fusion proteins and the like are included within the meaning of polypeptide.

The following examples are provided to illustrate certain embodiments of the invention. They are not intended to limit the invention in any way.

### Example I

AAV hu.32 capsid was cloned into an AAV2-based packaging plasmid to obtain a hybridconstruct with AAV2 rep and the alternative cap in frame as described (Gao et al. (2002) Proc. Natl. Acad. Sci., 99:11854-11859). All vectors comprised the cytomegalovirus promoter and enhanced GFP transgene and were cross-packaged into an AAV2 recombinant genome with heterologous cap sequence from the tested AAV variant using a triple-transfection procedure as described (Gao et al. (2002) Proc. Natl. Acad. Sci., 99:11854-11859). The packaging, purification, and determination of vector titers were performed by the University of Pennsylvania Vector Core. All recombinant vectors were purified using the CsCl sedimentation method and genome copy titers were determined as described (Gao et al. (2000) Hum. Gene Ther., 11:2079-2091).

Adult mice were injected intravenously with the hu.32 AAV vector comprising the GFP transgene. After injection, mice were anesthetized with a mixture of ketamine and xylazine (˜0.15 ml per mouse) and perfused transcardially with a solution of phosphate-buffered saline followed by 4% paraformaldehyde. Brains from animals were then removed and put in 4% paraformaldehyde overnight, following which they were transferred to 30% sucrose for cryoprotection. Once the brains sank in the sucrose, they were mounted in optimum cutting temperature solution (Sakura, Torrance, Calif.) and frozen at −20° C. until sectioning. Sectioning was done at a thickness of 20 μm using a cryostat (Leica Microsystems, Wetzlar, Germany) and the sections were mounted on three sets of slides which were then kept at −20° C. until imaging by confocal microscopy.

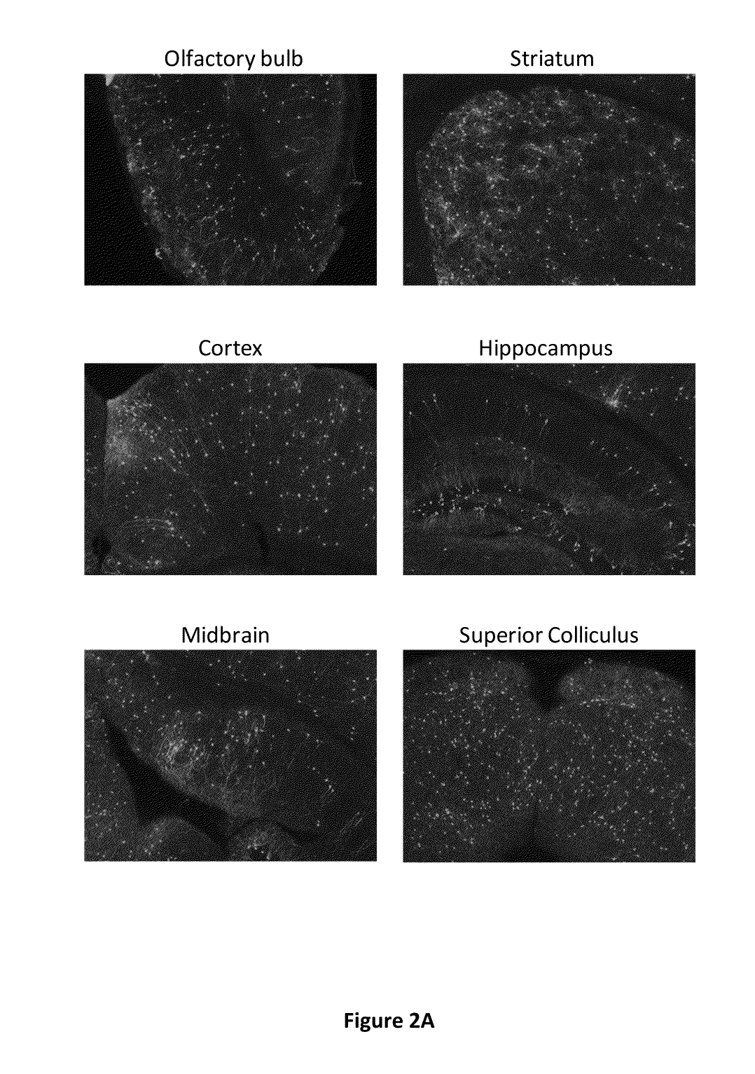

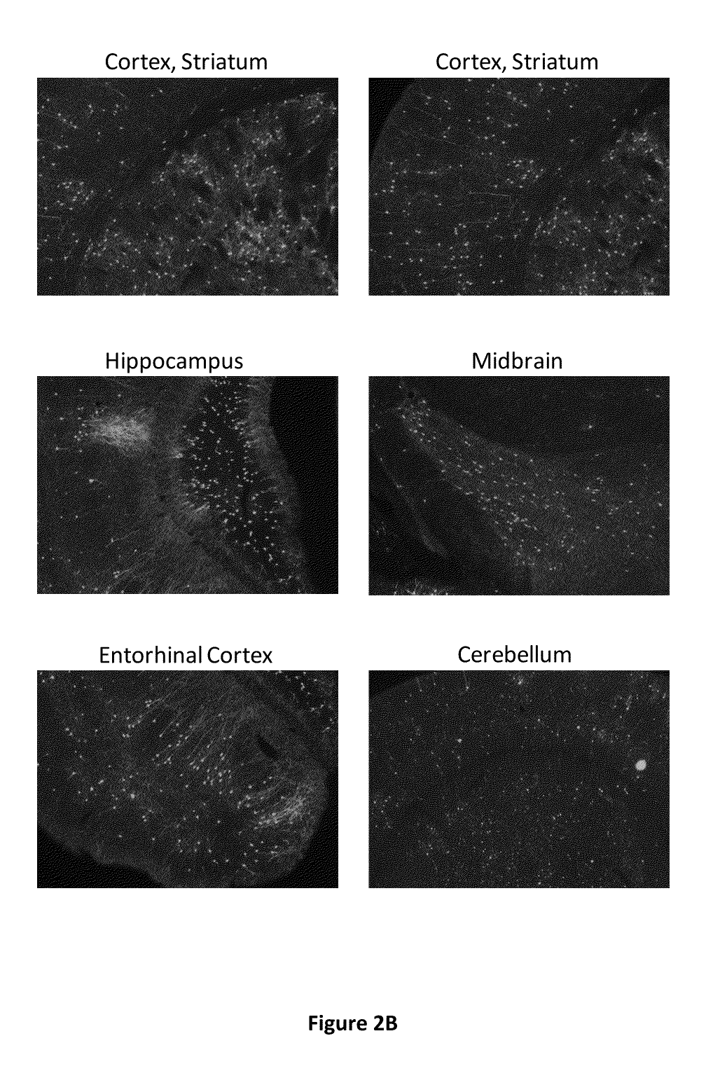

As seen in FIG. 2, GFP was expressed intensely throughout the brain after intravenous injection. More specifically, GFP expression was detected in neurons in the olfactory bulb, cortex, striatum, hippocampus, midbrain, superior colliculus, entorhinal cortex, and cerebellum. These results demonstrate substantially greater levels of transduction than observed with AAV9 (Foust et al. (2009) Nat. Biotechnol., 27:59-65). Further, the widespread expression of GFP has been observed in Balb/c, C3H, and C57B1/6 mice.

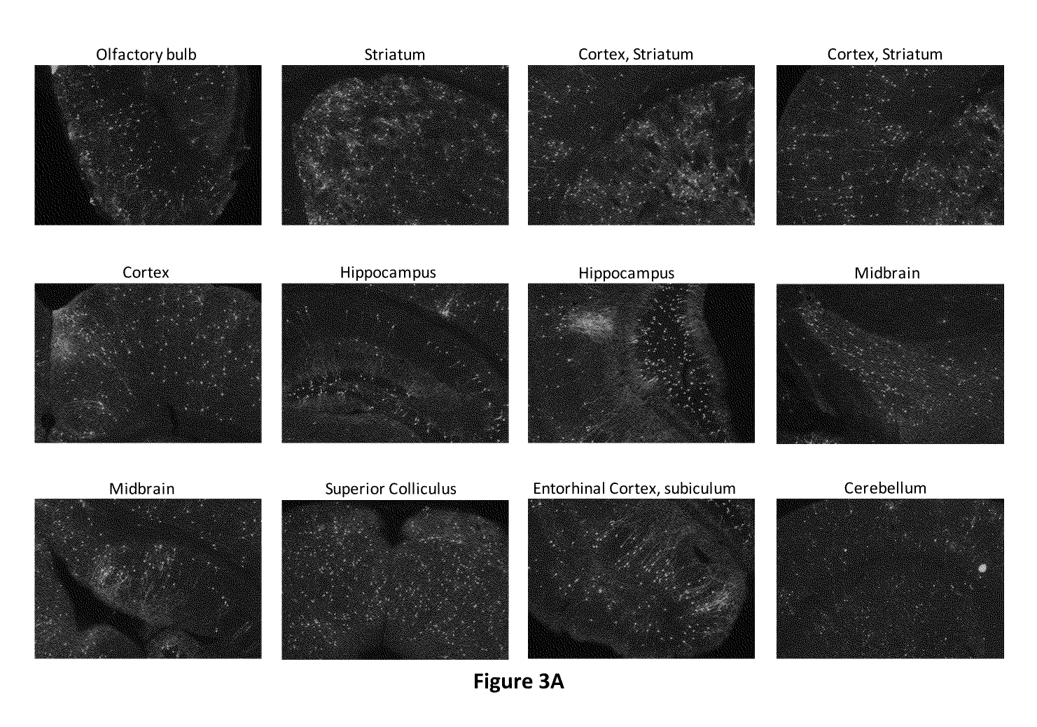

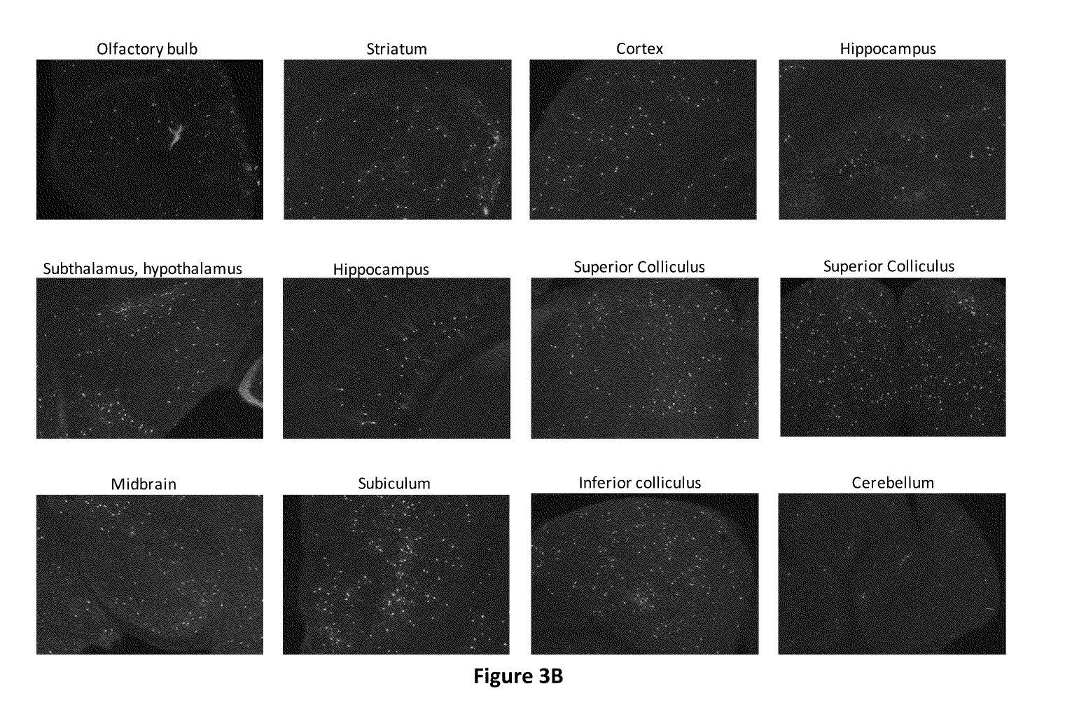

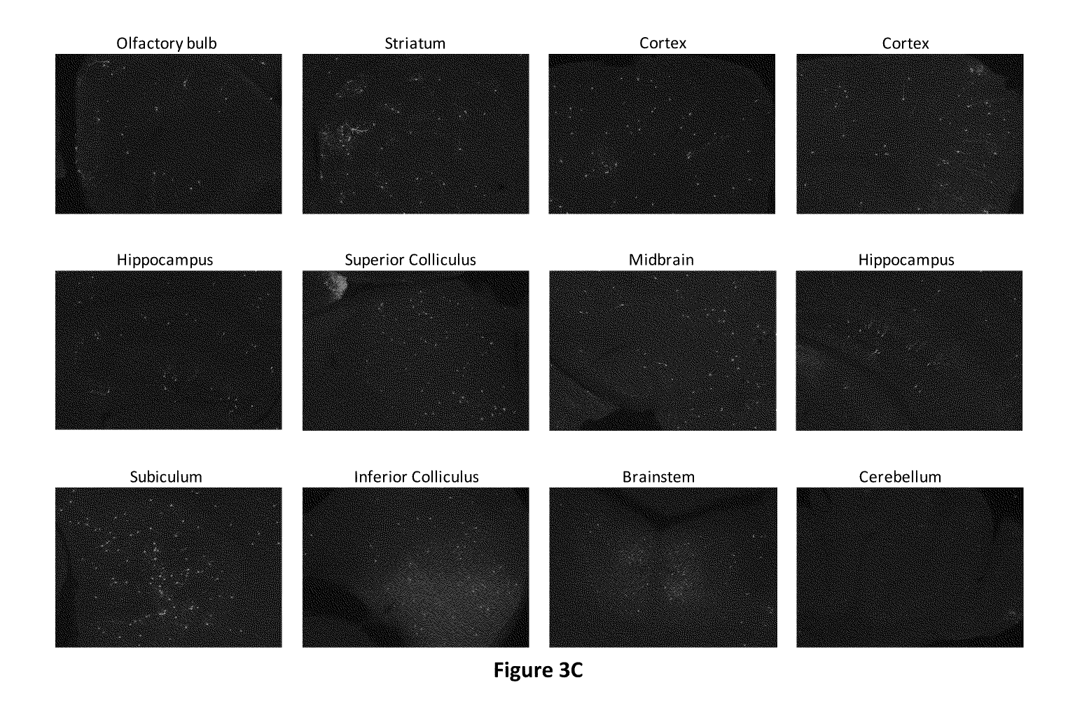

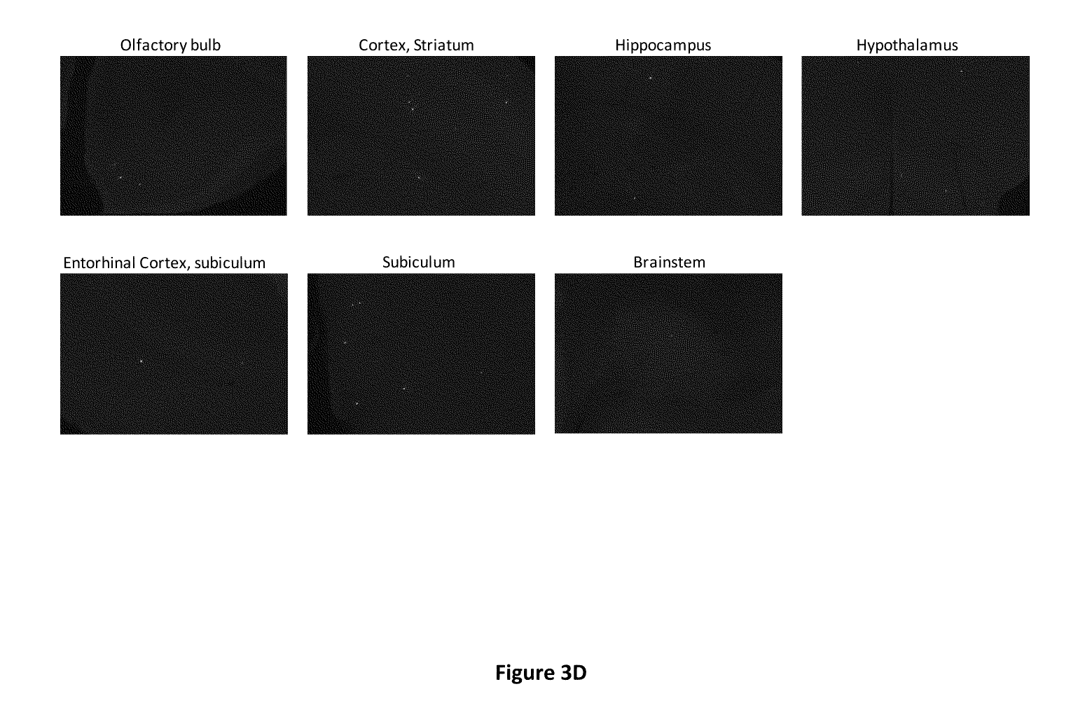

FIG. 3 shows a comparison of gene transfer for AAV2/9, AAV2/hull, AAV2/rh8, and AAV2/hu32. Mice were injected intravenously with the same quantity of virus. However, as evidenced by FIG. 3, hu32 dramatically increased the delivery to the brain over the other strains. Indeed, hull showed minimal targeting to the brain, AAV9 showed weak targeting, rh8 showed improved targeting, and hu32 showed unexpectedly robust targeting.

### Example II

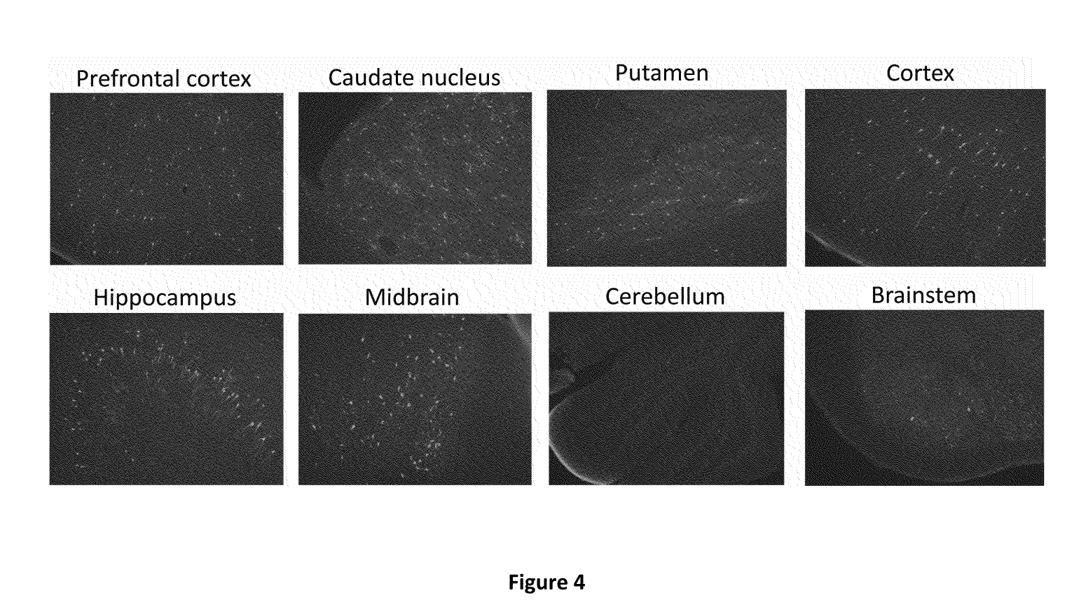

The targeting of the AAV vectors of the instant invention was also tested in cats. Six week old cats (n=3) were injected in the carotid artery with 2.88×10¹³ vector genomes (vg)/kg of AAV.hu32.hGBp.GFP, where hGBp is the human β-glucuronidase (hGUSB) promoter (378 bp fragment) and GFP is green fluorescent protein. GFP expression was monitored 8 weeks post-infection. As seen in FIG. 4, GFP was expressed intensely throughout the brain after intravascular (carotid) injection. More specifically, GFP expression was detected in neurons in the prefrontal cortex, caudate nucleus, putamen, cortex, hippocampus, midbrain, cerebellum, and brain stem.

### Example III

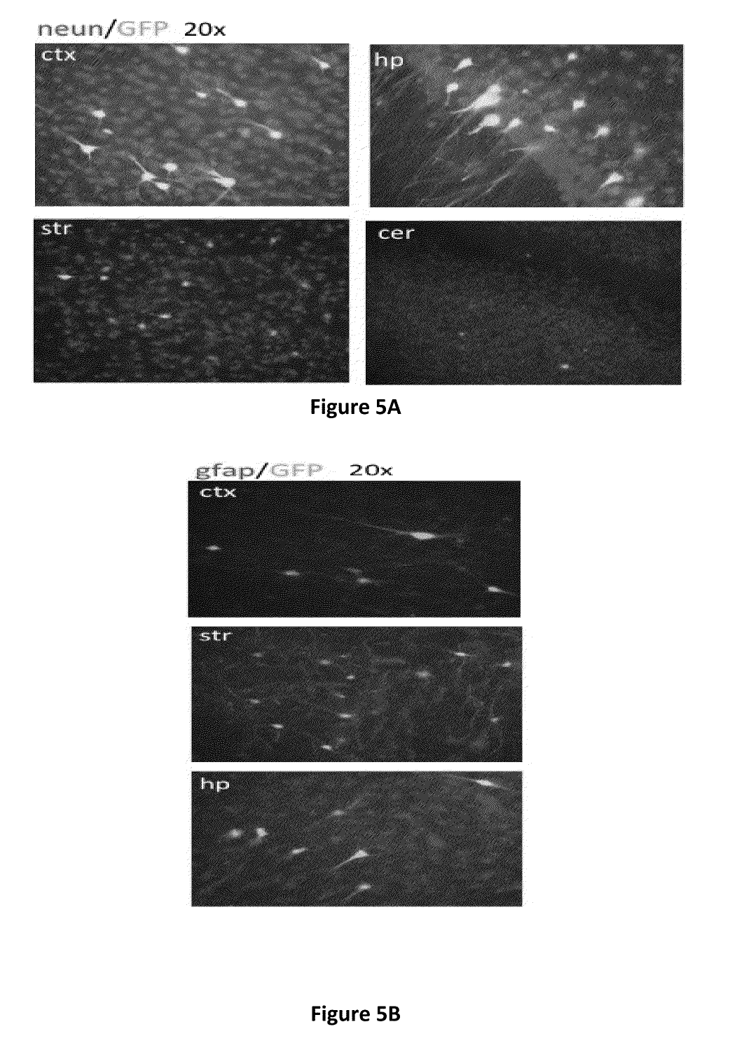

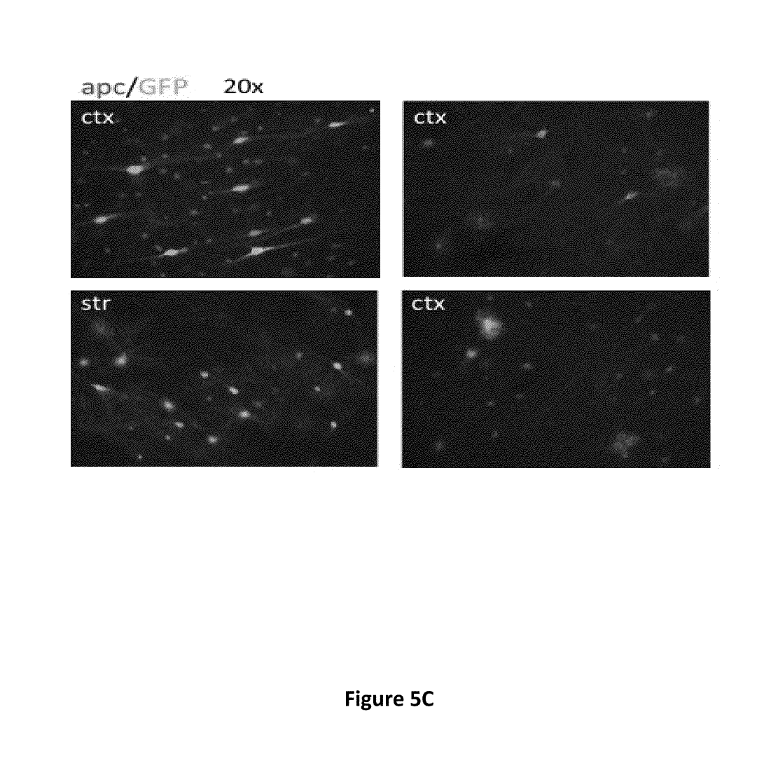

To demonstrate that the hu32 AAV vectors of the instant invention are infecting neurons, cells of infected brain regions were studied for GFP expression (indicating infection by the AAV vector) and cell-type specific markers. Specifically, expression of NeuN (Fox-3) was used to identify neurons, expression of glial fibrillary acidic protein (GFAP) was used to identify astrocytes, and expression of adenomatous polyposis coli (APC) was used to identify oligodendrocytes. FIG. 5A shows the double staining of neurons (GFP+, NeuN+) in the cortex, hippocampus, cerebellum, and striatum, indicating that the neurons were infected with GFP encoding hu32 AAV vector. In contrast, FIGS. 5B and 5C show that there is no double staining of astrocytes or oligodendrocytes, respectively, thereby indicating that the hu32 AAV vector did not transduce these cell types. Accordingly, these results demonstrate that the AAV vector of the instant invention is able to selectively infect neurons to the exclusion of astrocytes and oligodendrocytes.

### Example IV

Adeno-associated virus serotype 9 (AAV9) can cross the blood-brain barrier and infect neurons and astrocytes and other tissues (Foust et al. (2009) Nat Biotechnol., 27:59-65; Cearley et al. (2008) Mol. Ther., 16:1710-1718). However, it has recently been determined that AAV9 was unable to transduce CNS neurons in a mouse model of the lysosomal storage disease (LSD) mucopolysaccharidosis (MPS) VII (Chen et al. (2012) Mol. Ther., 20:1393-1399).

In stark contrast, the hu32 AAV vectors of the instant invention were capable of transducing neurons upon systemic administration. Table 1 shows β-glucuronidase (GUSB) activity of lysates of cryostat cut brain sections from 4 MPS VII mice treated with AAV.hu32.hGBp.GUSB. Briefly, GUSB enzyme activity was determined by the cleavage of a substrate to 4-methylumbelliferone (4-MU) by GUSB, where 4-MU can be detected fluorometrically. As seen in Table 1, the intravascular delivery of the hu32 AAV vector leads to transduction of brain neurons and very high—well above therapeutic levels—expression of GUSB.

TABLE 1
β-glucuronidase activity as percent of normal is provided
from 4 cryostat cut brain samples obtained from 4
MPS VII mice transduced with AAV.hu32.hGBp.GUSB.

| | nMoles/mg/hr | % normal |
|---|---|---|
| 24617 | 0.41 | 13.69 |
| 24734 | 0.31 | 10.45 |
| 24736 | 0.40 | 13.46 |
| 24740 | 0.44 | 14.82 |

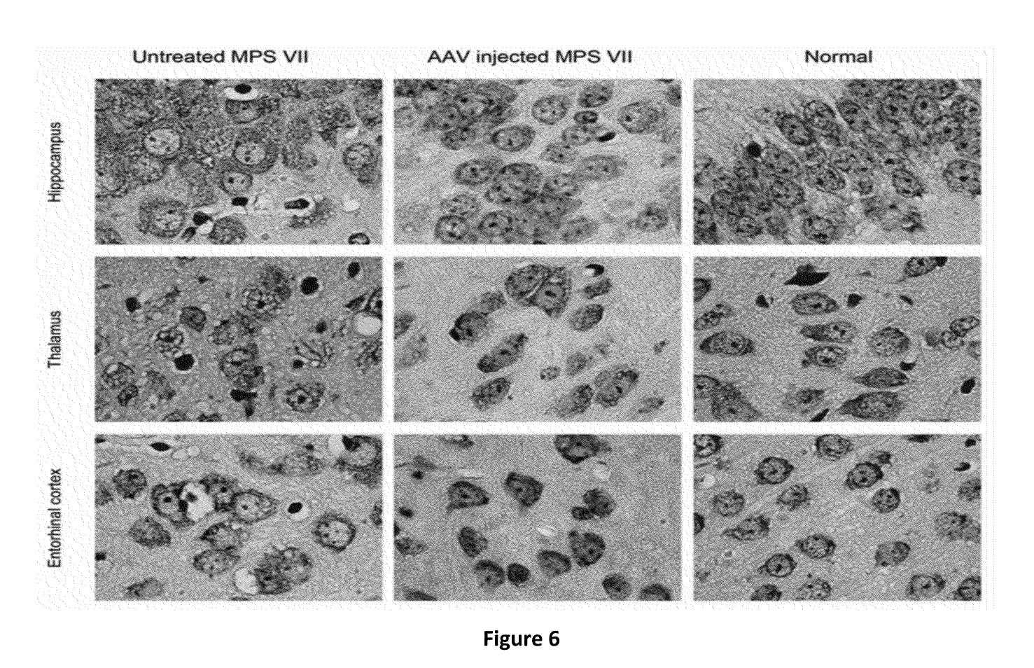

FIG. 6 provides histopathology images of normal mice, untreated MPS VII mice, and MPS VII mice transduced with AAV.hu32.hGUSB.GFP. Sections of the hippocampus, thalamus, and entorhinal cortex were examined. The untreated MPS VII mice brain slices show the characteristic lesions observed with MPS VII. In stark contrast, the MPS VII mice treated with AAV.hu32.hGBp.GUSB show a histopathology similar to normal mice without the hallmark lesions of MPS.

### Example V

A large number of single gene disorders affect the central nervous system (CNS), many of which are caused by deficiencies of specific proteins in metabolic pathways (Pierson et al. (2005) Neurogenetics: Scientific and Clinical Advances pp. 43-85, Marcel Dekker, New York). Somatic gene transfer can permanently correct the underlying metabolic deficiency by transferring a normal copy of a defective gene into a patient's own cells. Most metabolic disorders that affect the CNS produce lesions throughout the brain due to the fact that metabolic processes are shared by all cells or by cells of a specific type. In the brain, this means that the diseased cells are distributed globally and thus will require global, or at least widespread, correction mediated by widespread gene transduction.

Recently, AAV9 has gained attention due to their ability to cross the blood-brain barrier (BBB) and transduce neurons and astrocytes when injected intravenously in neonatal and adult animals (Bevan et al. (2011) Mol. Ther., 19:1971-1980; Duque et al. (2009) Mol. Ther., 17:1187-1196; Foust et al. (2009) Nature Biotech., 27:59-65; Gray et al. (2011) Mol. Ther., 19:1058-1069; Zhang et al. (2011) Mol. Ther., 19:1440-1448; Foust et al. (2010) Nature Biotech., 28:271-274; Ruzo et al. (2012) Human Gene Ther., 23:1237-1246; Fu et al. (2011) Mol. Ther., 19:1025-1033; Rahim et al. (2011) FASEB J., 25:3505-3518; Dominguez et al. (2011) Human Mol. Genet., 20:681-693; Samaranch et al. (2012) Human Gene Ther., 23:382-389; Valori et al. (2010) Sci. Transl. Med., 2:35ra42; Wang et al. (2010) Mol. Ther., 18:2064-2074). These vectors provide alternative means of transgene delivery to the CNS with a single noninvasive systemic injection and have been used to demonstrate therapeutic effects in animal models of CNS disorders. However, in large animal translational models, transduction is mostly restricted to glial cells and in the spinal cord. A very limited number of neurons are transduced. Furthermore, almost no gene transfer is seen in neurons of the cerebral cortex, which will be a crucial target region in many human diseases.

It has been shown with AAV9, the serotype used in the vast majority of intravenous delivery studies, that the genome could be transported to distal sites via axonal pathways (Cearley et al. (2008) Mol. Ther., 16:1710-1718; Cearley et al. (2006) Mol. Ther., 13:528-537; Cearley et al. (2007) J. Neurosci., 27:9928-9940). The ability of other serotypes to cross the BBB was investigated by injecting the vectors intravascularly and evaluating the transduction in the CNS. It is shown herein that AAVs hu.11, rh.8 and hu.32 were capable of transducing CNS when administered systemically. Hu.32 was the most efficient in a dose comparison study of intravenous injection in mice. Furthermore, hu.32 mediated very widespread transduction of the cerebral cortex in the cat and monkey brain, which has a gyrencephalic cerebral cortical structure. This study shows that systemic injection of hu.32 can deliver transgenes efficiently and mediate widespread neuronal transduction in the brain of adult mice, cats and monkeys. This study shows that hu.32 is an alternative vector that is more efficient for neuronal transduction following systemic injection and can be used for treatment of neurogenetic disorders.

### Methods

### Plasmid and AAV Production

GFP was cloned into the AAV packaging plasmid pZac2.1. The vector genome contained AAV2 terminal repeats, a human GUSB promoter, simian virus 40 splice donor/acceptor signal, bovine growth hormone polyadenylation signal. Recombinant AAVrh.8, AAVhu.32, AAVhu.11 and AAV9 were packaged following triple transfection of HEK293 cells by AAV cis-plasmid, AAV trans-plasmid containing AAVrep and cap genes and adenovirus helper plasmid. Vectors were purified using iodixanol gradient ultracentrifugation, and the titers were determined by real time PCR (Lock et al. (2010) Human Gene Ther., 21:1259-1271).

### Vector Injections

Normal BALB/c, C3H and B16 mice (8-12 week old) were used for experiments. Mice were injected into the tail vein with 200 μl of vector in phosphate buffered saline (PBS) at the indicated titers. Normal cats (6 week old) were used for cat experiments. Three cats were injected with AAVhu.32 vector expressing GFP at 2.9×10¹³ vg/kg dose into the common carotidartery. Three naive rhesus macaque monkeys were injected with 1.3×10¹³ vg/kg of AAVhu.32-GFP into the carotid artery. For the carotid injection of cats and monkeys, a catheter was placed in the cephalic vein and enough propofol was given to allow intubation. Animals were kept on anesthesia for the entire surgery. A small incision was made on the left side of the neck in order to expose the common carotidartery. A catheter was placed into the artery and flushed with saline. The vector was then infused and followed with more saline.

### Tissue Collection

Four weeks post-injection, mice were euthanized and transcardially perfused with 4% paraformaldehyde. Cats were euthanized at 6 weeks post-injection, and monkeys were euthanized at 8 weeks post-injection, transcardially perfused with PBS and the tissues were drop-fixed in 4% paraformaldehyde. Tissues were embedded in 2% agarose and sectioned coronally at 50 μm on a vibratome (Leica VT1000S, Leica, Buffalo Grove, Ill.). For serum collection, the whole blood was incubated for 30 minutes at room temperature followed by centrifugation at 1000 g for 15 minutes. The supernatant was then aspirated and stored at −80° C.

### Immunohistochemistry

GFP-positive cells were labeled and phenotyped using standard immunohistochemistry. Free-floating sections were permeabilized and immunoblocked for 30 minutes in 4% goat or donkey serum in PBS-T (PBS containing 0.3% Triton X-100). The sections were then incubated overnight at 4° C. with the following primary antibodies: rabbit anti-GFP (1:1000, Molecular Probes, Grand Island, N.Y.), mouse anti-NeuN (1:500, Millipore, Billerica, Mass.), chicken anti-GFAP (1:1,000, Millipore) and mouse anti-APC (1:100, Millipore). After three washes in PBS-T, sections were incubated with the appropriate fluorescently labeled secondary antibodies (1:250; Alexa 488 and Alexa 594, Molecular Probes) in PBS-T for 45 minutes. After removal of the secondary antibodies and further washes in PBS-T, the sections were mounted onto glass slides and cover-slipped with VECTASHIELD® Mounting Medium (Vector Laboratories, Burlingame, Calif.).

For DAB immunohistochemistry, blocking and primary antibody incubations were done as described above. Sections were washed in PBS-T and incubated with the appropriate biotinylated secondary antibodies (goat anti-rabbit, anti-mouse or anti-chicken, 1:250, Vector Laboratories) for 45 minutes followed by PBS-T washes. The antibody binding was visualized using VECTASTAIN® Elite ABC reagent and 3,3′-diaminobenzidine substrate kit for peroxidase (Vector Laboratories).

Sections were then mounted onto glass slides, dehydrated and mounted in Cytoseal™ 60 mounting medium (Richard Allen Scientific, Kalamazoo, Mich.) with glass coverslips. Images were visualized using a Leica AF6000 LX microscope (Leica, Heerbrugg, Switzerland) and acquired using a DFC 360FX or DFC 425 digital camera (Leica). GFP expressing cells were quantified in mouse brain hemi-sections at every 1 to 1.5 mm region. Images were converted to grey scale and the identical threshold was applied. The number of cells in the sections over the set threshold was counted by particle analysis using ImageJ software (NIH, Bethesda, Md.).

### Real Time PCR

Quantitative real time PCR was used to determine the viral genome copies present in the mouse brain and peripheral organs. For the brain, every 6th coronal section was pooled from each brain and the genomic DNA was extracted. Copies of GFP vector genome were quantified using LightCycler® FastStart DNA Master SYBR Green I mix (Roche, Indianapolis, Ind.) on a StepOne™ Real-Time PCR System (Applied Biosystems, Carlsbad, Calif.) and normalized to the GAPDH gene. For each gene assayed, triplicate samples derived from each DNA pool were used for quantification.

### Statistical Analysis

Unpaired two-tailed Student's t-test and One-Way ANOVA were used, where applicable, to determine whether mean differences between groups were different and were considered significant when P<0.05. Data are reported as means±SEM unless otherwise stated.

### Results

### Intravenous Injection of AAVhu.32 in Adult Mice Mediates Widespread Neuronal Transduction Throughout the Brain and Spinal Cord

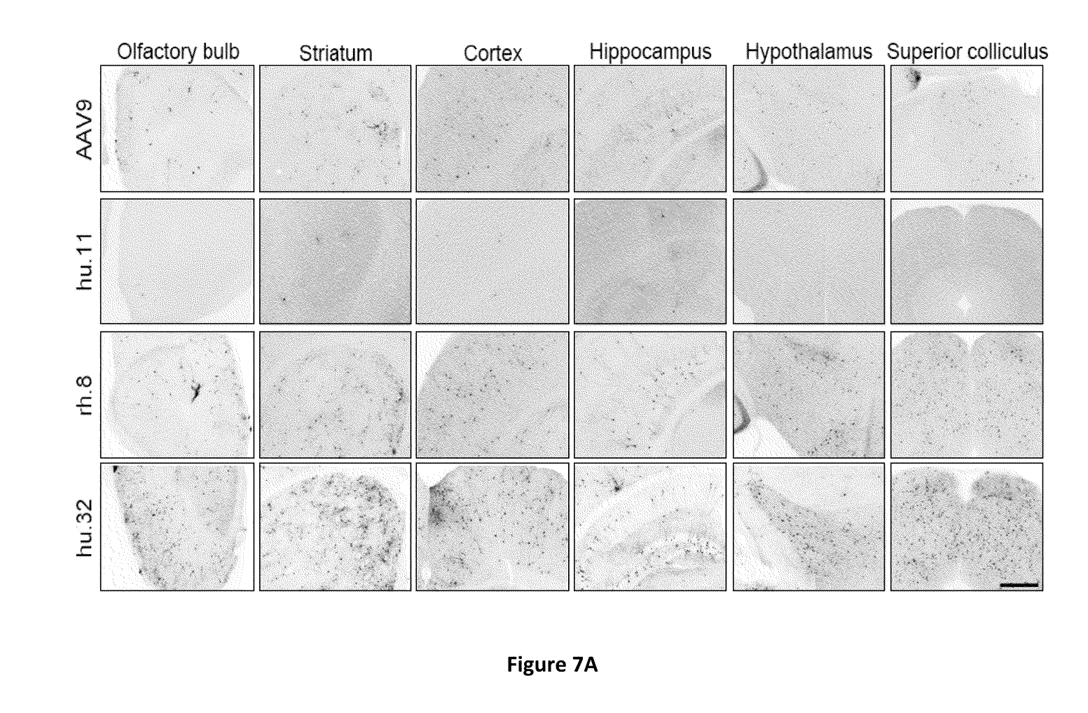

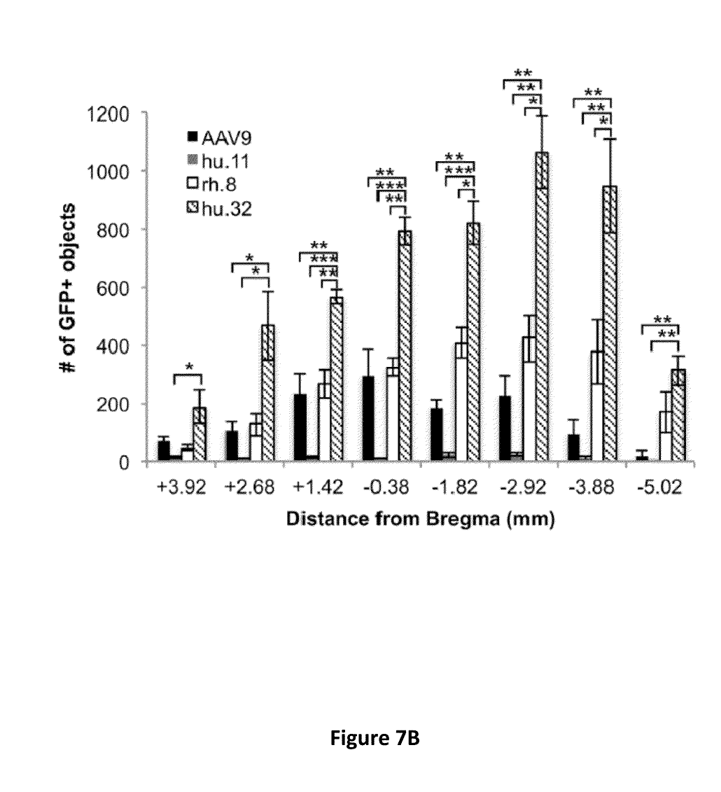

Novel AAV serotypes capable of neuronal transport (hu.11, rh.8 and hu.32) were compared to AAV9 for distribution of transduction in the mouse brain after injection into adult mice through the tail vein. For each AAV serotype, three age-matched (8-10 weeks) female BALB/c mice were injected with 200 μl of titer-matched vector (2.9×10¹² vectorgenomes (vg) total, 1.4×10¹⁴ vg/kg) encoding GFP and analyzed for GFP immunoreactivity 4 weeks post-injection. AAVhu.32 was the most efficient serotype and displayed the highest expression throughout the brain from the olfactory bulb to the cerebellum, almost exclusively in the gray matter (FIG. 7A). This was followed by rh.8 and AAV9, which displayed similar patterns of transduction. Hu.11 exhibited the lowest level of transduction compared to other serotypes examined.

The amount of transduction was quantified and hu.32 had the highest number of GFP positive cells throughout the brain (FIG. 7B), consistent with the GFP expression observed by immunofluorescence. Higher transduction was observed in the caudal part of the brain. The vector genomes present in the brain were also quantified by qPCR using the genomic DNA extracted from pooled coronal sections of the brain. In general, the distribution of vector genomes in the brain was correlated with the GFP expression seen by immunofluorescence. Hu.32 had approximately 2-fold more vector genome transported to the brain than AAV9 (Table 2).

TABLE 2
Vector genome copies in the brain of mice
4 weeks following intravenous injection.

| Serotype | n | mean | SEM | P vs. hu.32 |
|---|---|---|---|---|
| PBS | 2 | 0.00 | 0.00 | 0.0537 |
| AAV9 | 3 | 2.22 | 0.83 | 0.1633 |
| hu.11 | 3 | 0.26 | 0.07 | 0.0197 |
| rh.8 | 3 | 2.09 | 0.66 | 0.1274 |
| hu.32 | 3 | 4.67 | 1.17 | — |

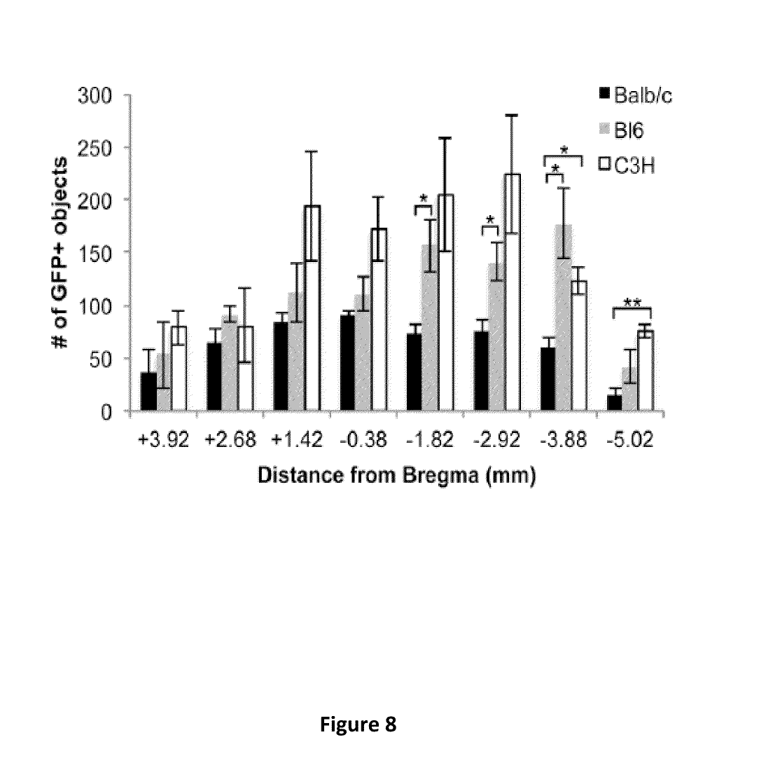

CNS transduction was assessed in BALB/c, C3H and B16 mice to test whether the same pattern and level of transduction occurred in different strains of mice. The transduction efficiency in the brain was assessed by counting the number of GFP-positive objects. AAVhu.32 exhibited higher levels of transduction in C3H, followed by B16 and BALB/cmice, but the pattern of transduction with respect to brain structures was similar in all 3 strains (FIG. 8).

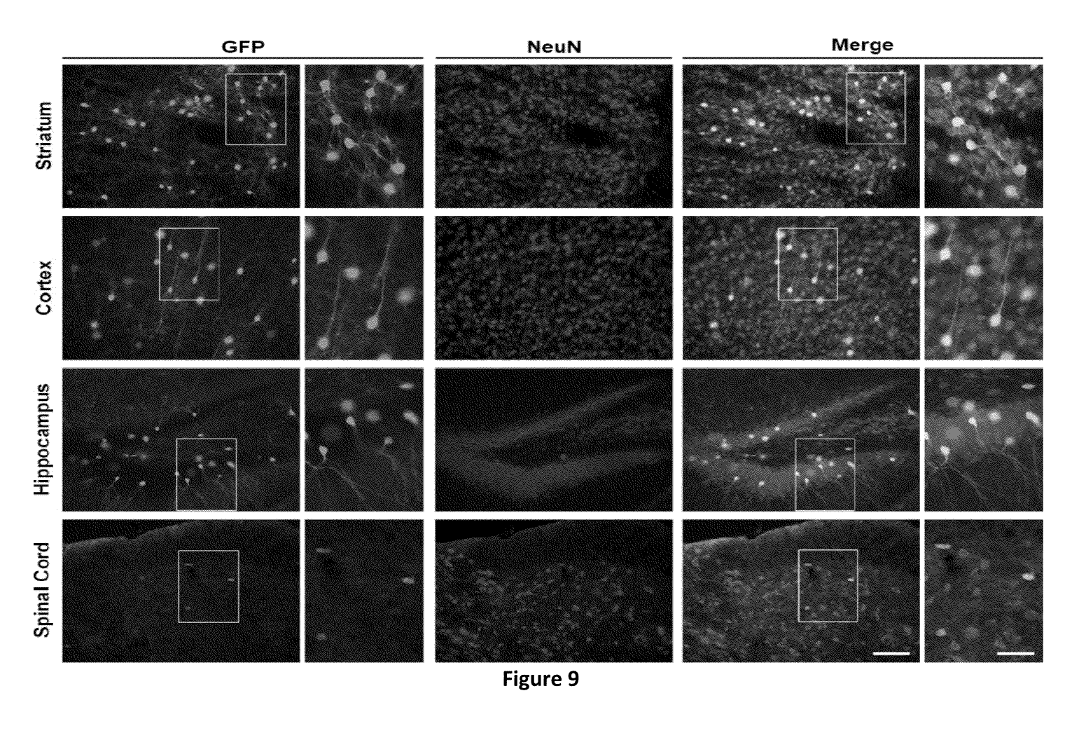

The phenotypes of the transduced cells in the brain were analyzed by double immunofluorescent staining. In the brain, transduced cells were predominantly neurons by morphology and this was verified by dual immunofluorescent staining with antibodies against GFP and a neuronal marker, NeuN in the striatum, cortex and hippocampus (FIG. 9). Dual staining with GFAP or APC did not reveal colocalization of these markers in transduced cells. Transduction was seen in various morphologic types of neurons throughout the brain and the transduction appeared to be non-preferential. In the spinal cord, GFP-positive cells were also predominantly neuronal by morphology and they co-stained with anti-NeuN antibody (FIG. 9).

### Distribution of AAV Vector Genome in Peripheral Organs Following Intravenous Injection of AAVhu.32 in Adult Mice

Since a potential limitation of intravenous vector delivery is the high degree of vector delivery to peripheral organs outside the CNS, the amount of vector genome in the peripheral organs was quantified by qPCR. The liver was highly transduced, whereas minimal levels of vector genome were detected in the heart, kidney and spleen (Table 3). Significant differences in peripheral tissue tropism were not observed compared to AAV9. At 4 weeks following intravenous injection of 1×10¹¹ or 1×10¹² vg of AAVhu.32-GFP in mice, blood was collected to measure serum levels of blood urea nitrogen (BUN), albumin and alanine amino transferase (ALT) to evaluate kidney, general inflammation and liver function, respectively. Overall, AAVhu.32-GFP did not cause any significant changes in BUN, albumin or ALT when compared to the levels in uninjected mice (Table 4).

TABLE 3
Vector genome copies in the peripheral organs of mice 4 weeks following
intravenous injection.

| Serotype | n | mean | SD | P vs. hu.32 | mean | SD | P vs. hu.32 |
|---|---|---|---|---|---|---|---|
| | | Liver | | | Heart | | |
| PBS | 2 | 0.08 | 0.12 | — | 0.03 | 0.01 | — |
| AAV9 | 2 | 40.27 | 17.93 | 0.7892 | 0.10 | 0.14 | 0.4667 |
| hu.11 | 2 | 0.03 | 0.04 | 0.0006 | 0.01 | 0.01 | 0.4287 |
| rh.8 | 2 | 37.55 | 5.92 | 0.9587 | 1.17 | 0.04 | 0.855 |
| hu.32 | 2 | 36.40 | 1.23 | — | 0.96 | 1.36 | — |
| | | Kidney | | | Spleen | | |
| PBS | 2 | 0.03 | 0.04 | — | 0.02 | 0.02 | — |
| AAV9 | 2 | 0.10 | 0.13 | 0.1842 | 0.08 | 0.08 | 0.439 |
| hu.11 | 2 | 0.02 | 0.02 | 0.1334 | 0.03 | 0.03 | 0.4276 |
| rh.8 | 2 | 0.31 | 0.09 | 0.3393 | 0.38 | 0.49 | 0.5322 |
| hu.32 | 2 | 0.63 | 0.35 | — | 1.59 | 2.24 | — |

TABLE 4
Mouse serum levels of blood urea nitrogen, albumin and alanine
amino transferase following AAVhu.32 intravenous injection.

| | | Blood Urea Nitrogen | Albumin | ALT |
|---|---|---|---|---|
| Dose | n | (mg/dL) | (g/dL) | (U/L) |
| uninjected | 3 | 13.3 ± 6.4 | 4.7 ± 0.6 | 20.0 ± 12.0 |
| 1E+11 vg | 3 | 19.7 ± 4.0 | 5.9 ± 2.0 | 35.7 ± 7.0 |
| 1E+12 vg | 2 | 22.0 ± 5.7 | 7.5 ± 2.9 | 48.0 ± 55.2 |
| Reference range | | 18-29 | 2.5- 4.8 | 28-132 |

### CNS Transduction in Cats by Intracarotid Delivery of AAVhu.32

The efficient neuronal transduction in the adult mice following intravenous injection of AAVhu.32 prompted further evaluation of the serotype in large animals. Differences in the patterns of CNS transduction have been observed in previous studies in large mammals with lower overall transduction efficiency compared to mice. Notably, most of the transduction in the CNS of large animals has occurred in the spinal cord, with only small amounts present in the brain and limited in brain structures.

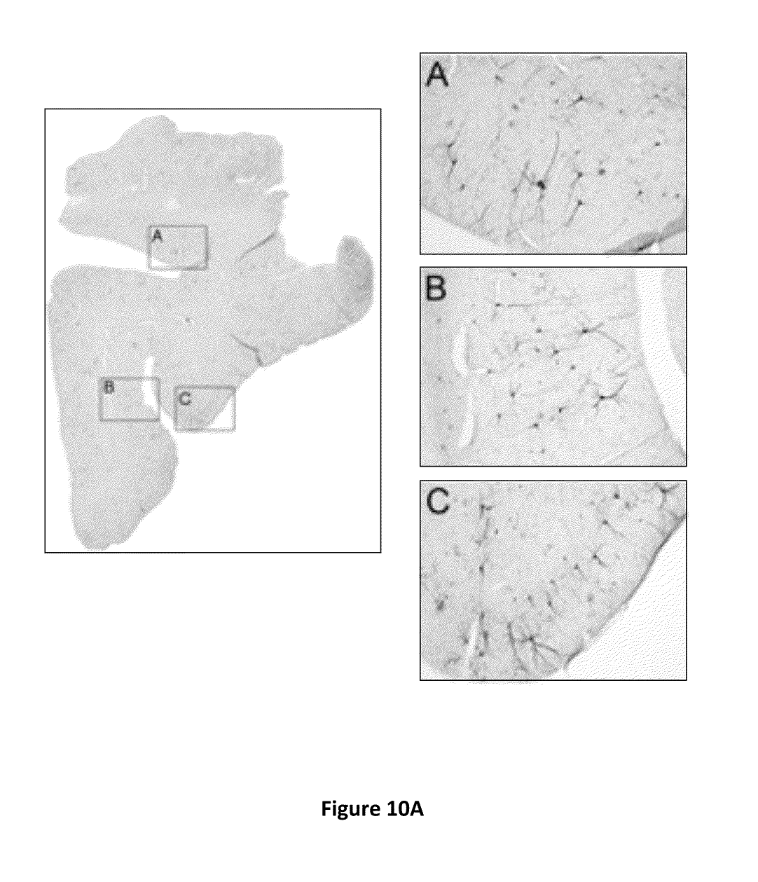

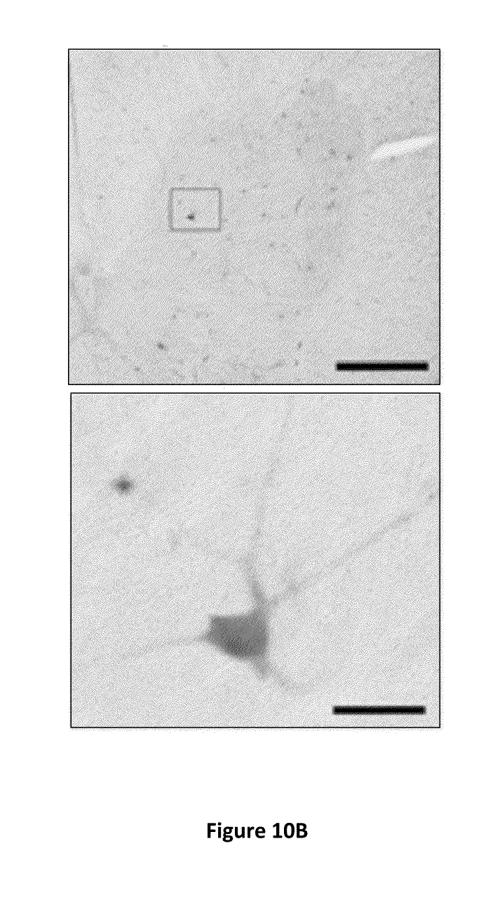

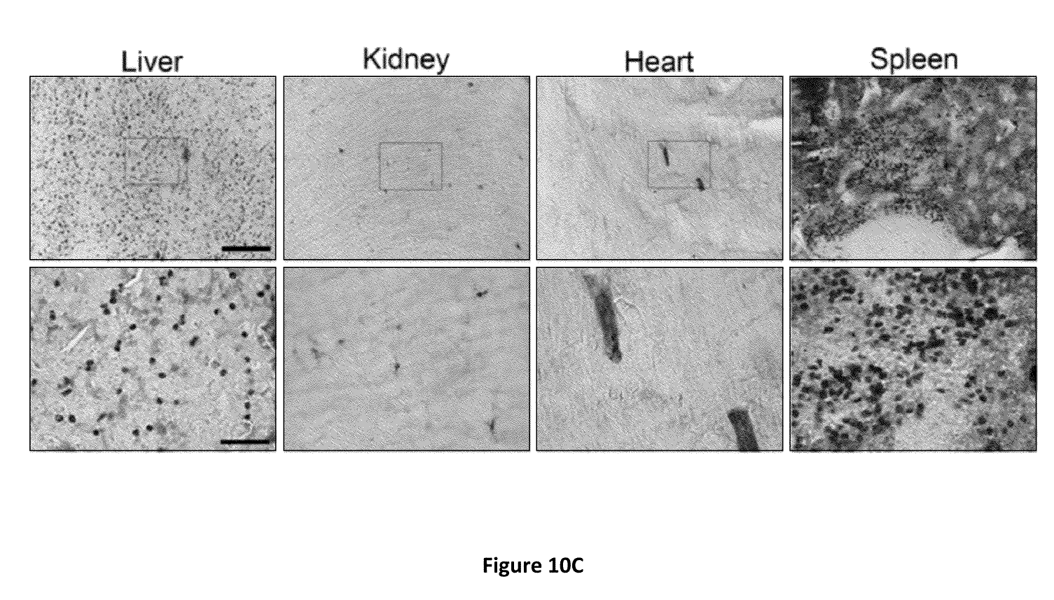

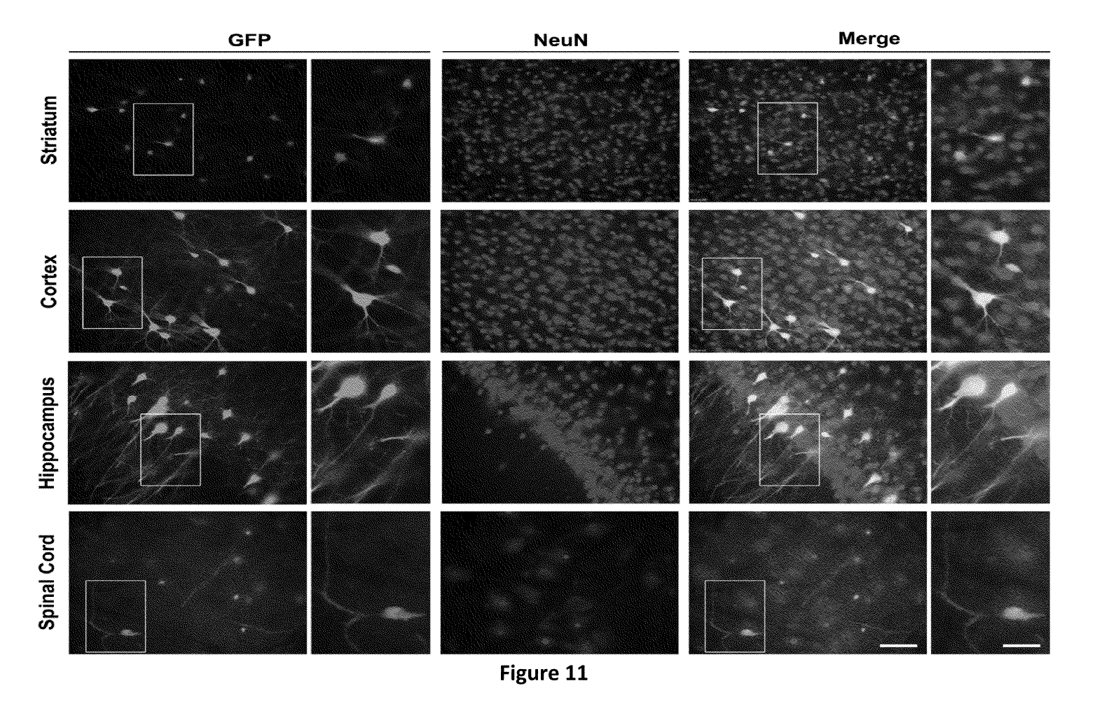

AAVhu.32 was tested for the ability to transduce the CNS following injection of 2.9×10¹³ vg/kg of AAVhu.32-GFP into the carotid artery of three 6-week-old cats. All the cats recovered well after the procedure. Serum chemistry at 6 weeks post-injection were within or near the reference range except for one cat that displayed an increase in BUN/Creatinine ratio, ALT and AST (Table 5). At 6 weeks post-injection, cats were euthanized and vector transduction was analyzed throughout the brain by immunohistochemistry. In the cat brain, AAVhu.32 transduced both gray and white matter regions, although the majority of transduced cells were of neuronal morphology in the gray matter (FIG. 10). The cortex, caudate nucleus, putamen, hippocampus and midbrain in particular were highly transduced. Most types of neurons were transduced throughout the brain and the transduction appeared to be non-preferential. In addition to the neuronal transduction, cells with astrocyte and oligodendrocyte morphology were transduced in the brain. In the spinal cord of transduced cats, GFP-positive cells were predominantly oligodendrocyte-like cells, based on their morphology. Neuronal transduction in the brain and spinal cord were confirmed by colocalization of GFP with NeuN (FIG. 11). Double immunofluorescence labeling with anti-GFP and anti-GFAP, anti-APC or anti-Choline acetyltransferase (ChAT), a motor neuron marker showed no colocalization of these markers in transduced cells. Vector transduction in the peripheral organs was also analyzed by immunohistochemistry. The liver and spleen were highly transduced, whereas the kidney and heart expressed very low levels of GFP (FIG. 10C).

TABLE 5
Cat serum chemistry following AAVhu.32 intracarotid injection.

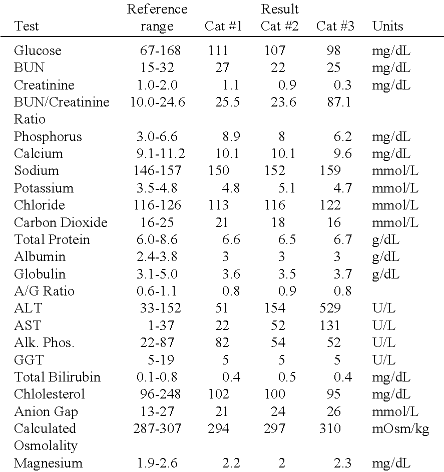

### CNS Transduction in Monkeys by Intracarotid Delivery of AAVhu.32

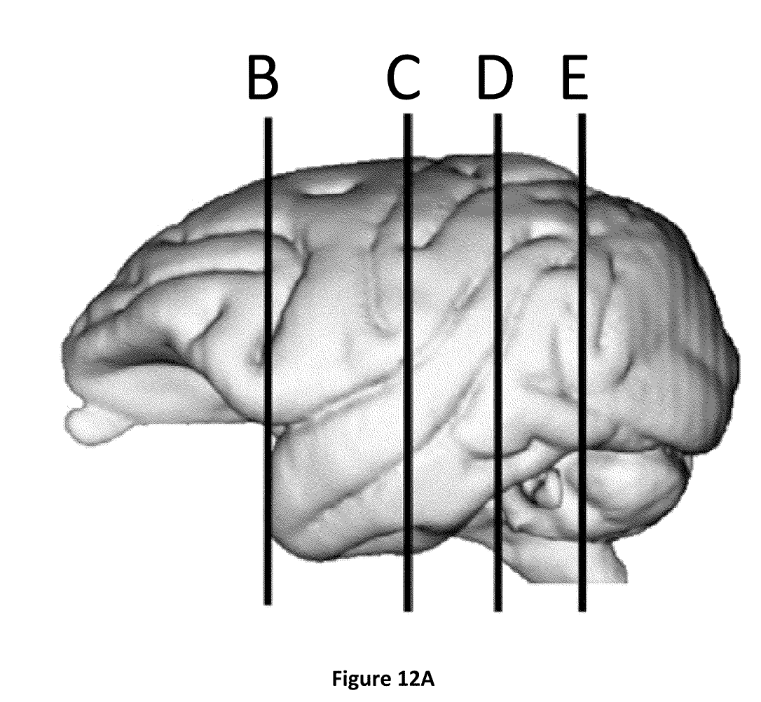

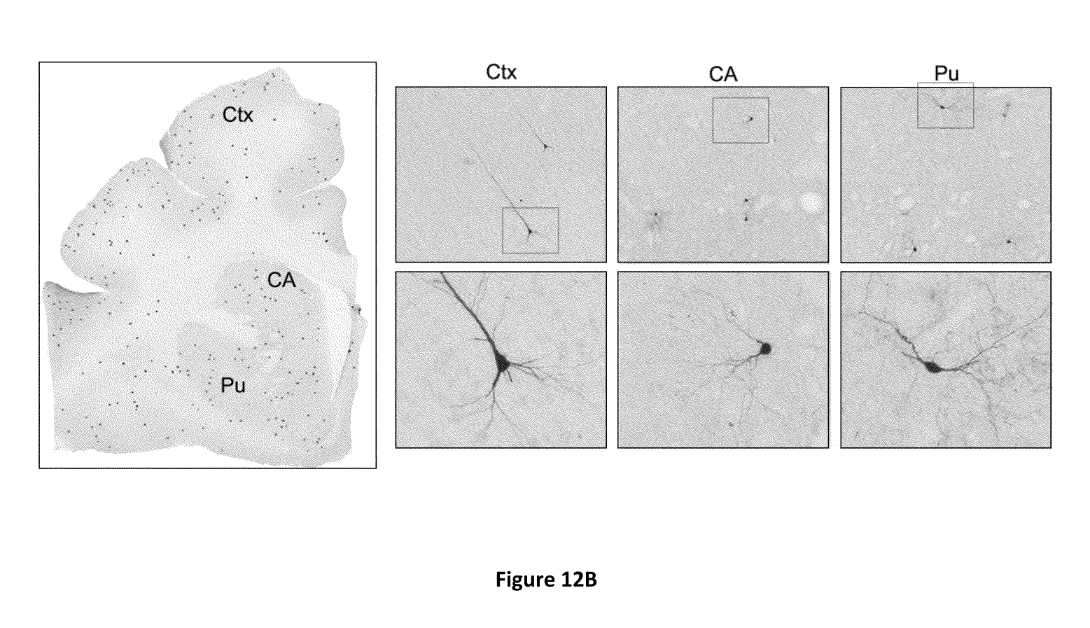

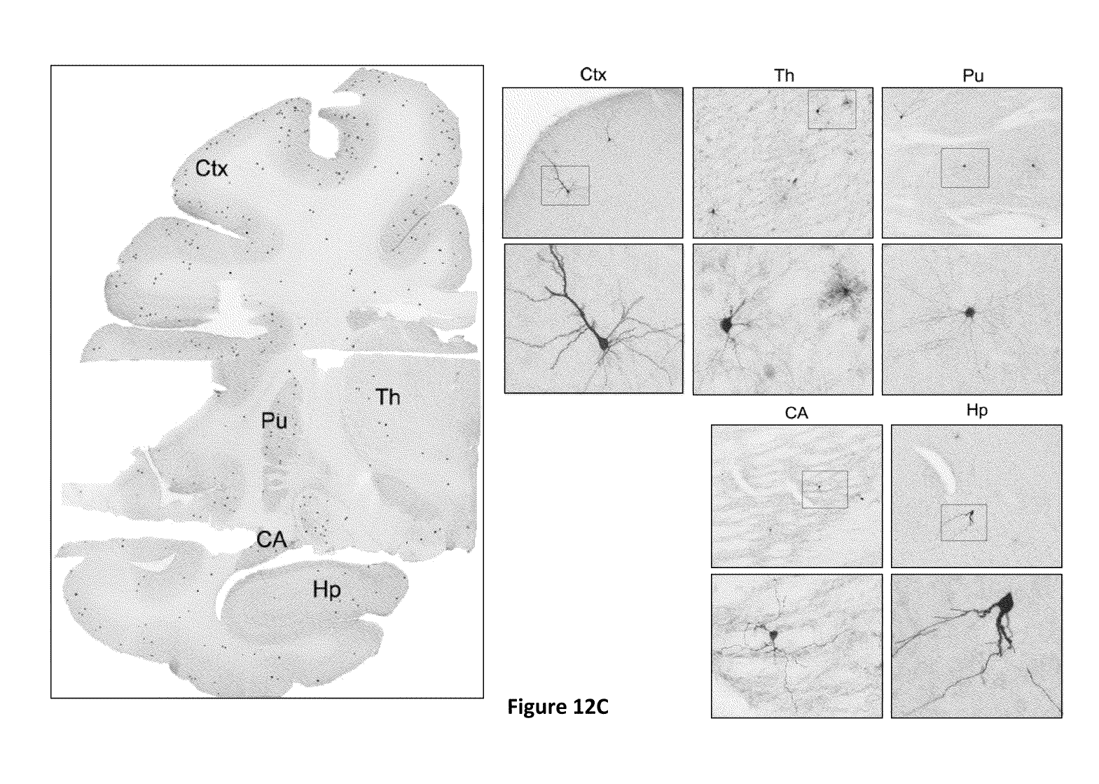

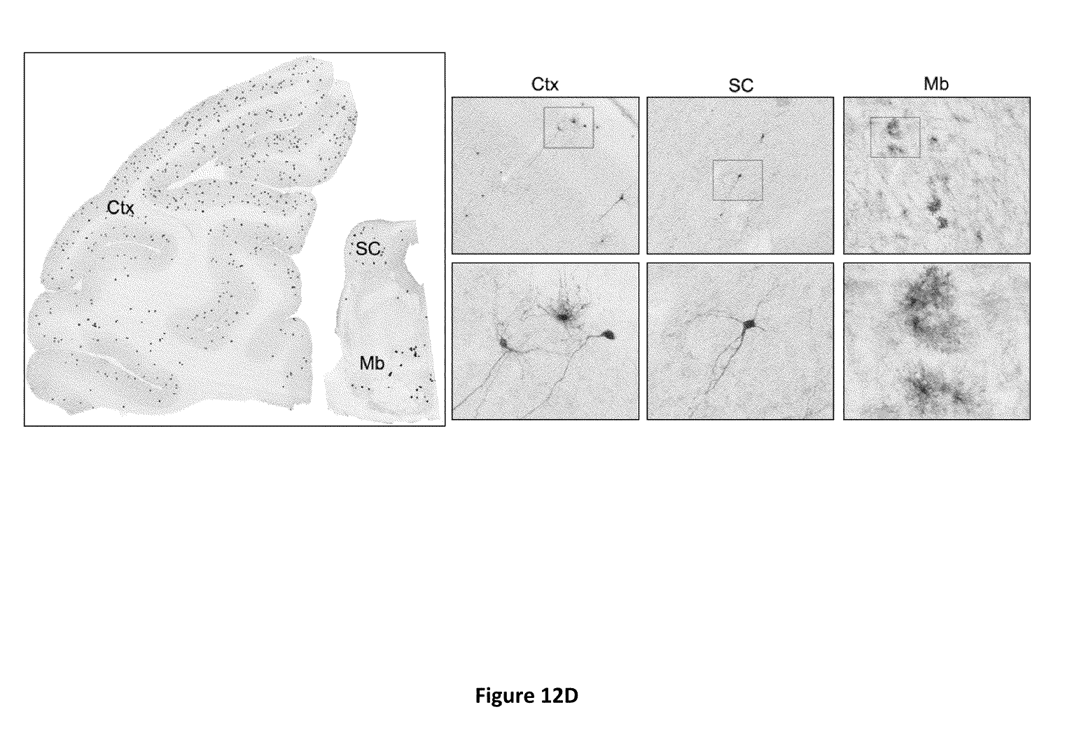

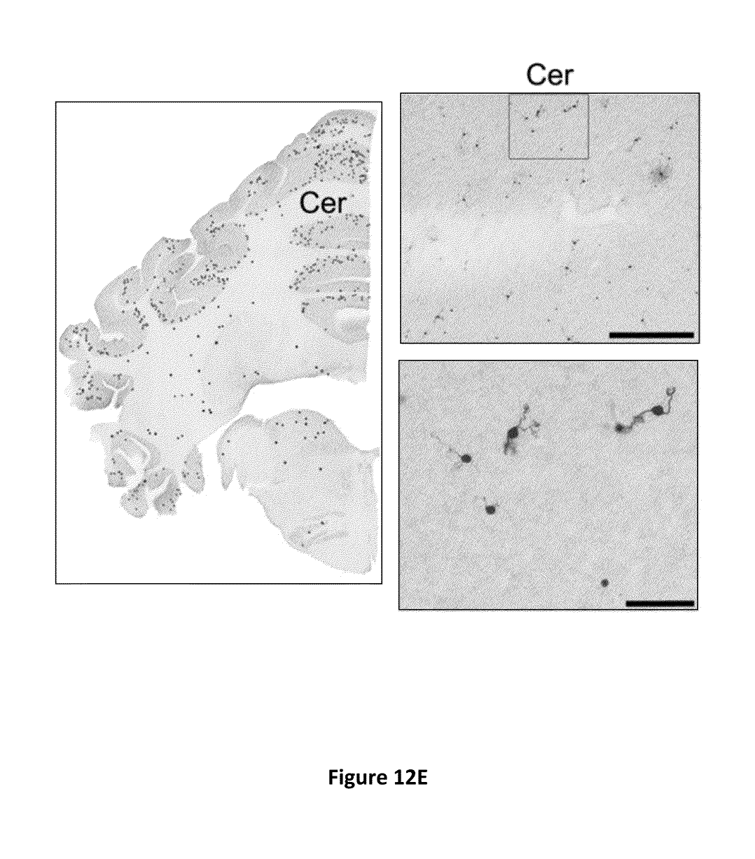

To further evaluate the clinical translation ability of AAVhu.32, three monkeys were injected with 1.3×10¹³ vg/kg of AAVhu.32-GFP into the carotid artery and vector transduction was analyzed by immunohistochemistry at 8 weeks post-injection (FIG. 12). The monkeys were widely transduced throughout the brain. As in the cat brain, AAVhu.32 transduced both gray and white matter regions in the monkey brain. The cortex, caudate nucleus, putamen and cerebellum were highly transduced with GFP positive cells also present in the hippocampus, thalamus and midbrain. Based on morphology, most GFP-expressing cells in the brain were neurons with some glial cells in the white matter regions. GFP-positive neurons outnumbered the glial cells by a ratio of 5.6 to 1 (Table 6).

TABLE 6
GFP-positive neuron to glia ratio in monkey brain.

| | Monkey #1 | | Monkey #2 | | Monkey #3 | | mean |
|---|---|---|---|---|---|---|---|
| section | neuron | glia | neuron | glia | neuron | glia | neuron/glia |
| 1 | 51 | 17 | 56 | 16 | 212 | 52 | 3.5 |
| 2 | 83 | 23 | 152 | 24 | 291 | 76 | 4.6 |
| 3 | 114 | 20 | 168 | 40 | 538 | 213 | 4.1 |
| 4 | 169 | 15 | 210 | 20 | 784 | 92 | 10.1 |

| | |
|---|---|
| mean | 5.6 |

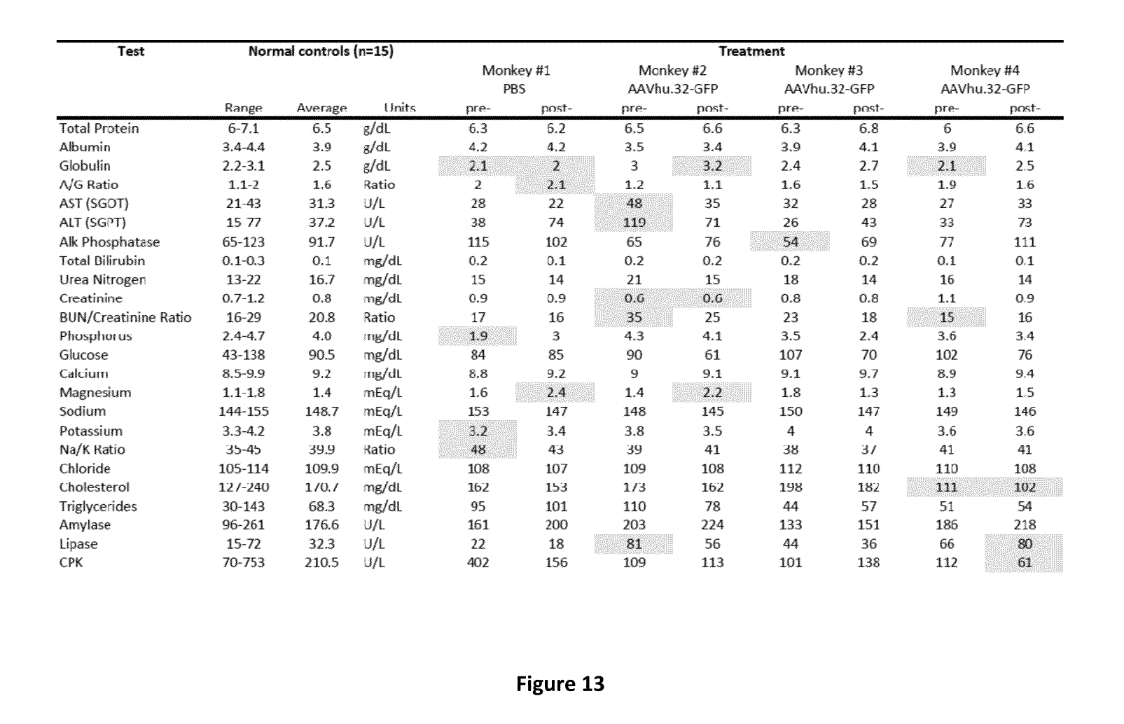

Clinical chemistry assays were performed pre-injection and at 8 weeks post-injection. None of the vector-injected animals had any serum chemistry value outside the range of normal age-matched control monkeys from the colony (FIG. 13). A few of the pre-injection values were slightly outside the control range, but were within the normal range at the end of the experiment. Thus, there was no indication for any liver, renal or other toxicity from the vector injections in the monkeys.

Intravascular delivery of AAV to the brain is clinically relevant for a number of diseases affecting the brain as it allows global gene transfer with a minimally invasive procedure. Certain AAV serotypes, including AAV9, have been described to be capable of crossing the BBB and mediate CNS gene delivery when administered systemically into mice. However, these AAV serotypes demonstrate significantly reduced brain transduction efficiency and primarily glial transduction in brain and spinal cord of large animals following systemic administration (Bevan et al. (2011) Mol. Ther., 19:1971-1980; Duque et al. (2009) Mol. Ther., 17:1187-1196; Gray et al. (2011) Mol. Ther., 19:1058-1069; Foust et al. (2010) Nat. Biotechnol., 28:271-274; Samaranch et al. (2012) Hum. Gene Ther., 23:382-389). Furthermore, almost no gene transfer is seen in neurons of the cerebral cortex, which will be a crucial target region in many human diseases. In stark contrast, it is shown herein that AAVhu.32 is capable of transducing predominantly neurons in a widely distributed pattern throughout the brain when injected intravascularly into cats and monkeys.

All of the previous large animal studies have used self-complementary AAVs (ssAAV) as they have higher transduction efficiency than traditional single-stranded vectors in mice (Gray et al. (2011) Mol. Ther., 19:1058-1069; McCarty, D. M. (2008) Mol. Ther., 16:1648-1656; McCarty et al. (2003) Gene Ther., 10:2112-2118). However, the packaging capacity of the scAAVs is approximately half that of conventional single-stranded AAVs, limiting their use for many therapeutic genes. This also significantly limits the amount of transcriptional control sequences that can be used to achieve cell-type specific expression if desired. In the present study, a single-stranded AAV genome packaged in the hu.32 cap vector was able to achieve robust widespread transduction of the CNS. Using an ssAAV vector with the larger packaging capacity enables a greatly expanded repertoire of gene therapy for the CNS, for example, cDNA coding sequences greater than about 2 kb.

Without being bound by theory, the superior transduction efficiency of AAVhu.32 among the serotypes investigated is likely due to different vector biology between different serotypes and differences in cell tropism and vector uptake. The fact that the choroid plexus was highly transduced suggests that hu.32 may enter the CNS by exploiting the extensive vasculature and fenestrated capillaries in circumventricular organs including the choroid plexus (Duvernoy et al. (2007) Brain Res. Rev., 56:119-147). Another potential route may be through direct transcytosis via endothelia of blood vessels, which has been shown with AAV in vitro (Di Pasquale et al. (2006) Mol. Ther., 13:506-516).

No adverse clinical effects were observed in any of the animals following intravascularvector injection. Serum chemistries of the vector-injected animals showed values within or near the reference range except for one cat that displayed an increase in BUN/Creatinine ratio, ALT and AST. Others have reported transient rise in ALT levels, inflammation and immune responses following intravascular or intrathecal GFP injection (Gray et al. (2011) Mol. Ther., 19:1058-1069). Variability between animals could also be attributed to serum hemolysis and animal dehydration, which can cause artifactual increases in BUN and albumin (Banks et al. (1996) J. Amer. Vet. Med. Assoc., 209:1268-1270; Lippi et al. (2006) Clin. Chem. Lab. Med., 44:311-316).

The finding of widespread transduction of the brain in all of the vector-injected animals was consistent with lack of antibodies to hu32, as pre-existing AAV9 neutralizing antibodies have been associated with low CNS transduction after systemic injection in large animals (Gray et al. (2011) Mol. Ther., 19:1058-1069; Samaranch et al. (2012) Hum. Gene Ther., 23:382-389). Epidemiological data shows a low prevalence of neutralizing antibodies against AAV serotypes other than 2 (Boutin et al. (2010) Hum. Gene Ther., 21:704-712; Calcedo et al. (2009) J. Inf. Dis., 199:381-390; van der Marel et al. (2011) Inflam. Bowel Dis., 17:2436-2442; Calcedo et al. (2011) Clin. Vaccine Immun., 18:1586-1588). Furthermore, the prevalence of anti-AAV antibodies in infants and young children is low (Calcedo et al. (2011) Clin. Vaccine Immun., 18:1586-1588; Chen et al. (2005) J. Virol., 79:14781-14792; Erles et al. (1999) J. Med. Virol., 59:406-411), which favors AAV gene therapy in children.

The widespread cerebral cortical neuronal transduction pattern of AAVhu.32 has important implications for treating many disorders of the CNS. Most neurogenetic diseases and neurodegenerative disorders result in pathological changes throughout the cerebral cortex. While treatment may depend on having a rational molecular target for modification, such diseases as lysosomal storage diseases, Alzheimer's disease, Huntington's disease, or amyotrophic lateral sclerosis have significant involvement of the cerebral cortex. In addition, with cell type specific promoters, the vector could also be used in diseases where expression is only required in restricted regions. Finally, this provides a means to deliver genes into the cerebrum in higher mammals for experimental manipulations, such as optogenetics, without the confounding effects of neurosurgery.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for delivering a nucleic acid molecule to the brain of a subject, said method comprising administering an adeno-associated virus (AAV) vector to said subject, wherein said AAV vector comprises said nucleic acid molecule and comprises hu.32 capsid protein or rh.8 capsid protein.

2. The method of claim 1, wherein said AAV vector comprises hu.32 capsid protein.

3. The method of claim 1, wherein said capsid protein comprises an amino acid sequence having at least 90% identity with SEQ ID NO: 1 or 3.

4. The method of claim 1, wherein said capsid protein comprises an amino acid sequence having at least 95% identity with SEQ ID NO: 1.

5. The method of claim 1, wherein said capsid protein comprises SEQ ID NO: 1.

6. The method of claim 1, wherein said nucleic acid molecule encodes a therapeutic protein or inhibitory nucleic acid molecule.

7. The method of claim 1, wherein said nucleic acid molecules are delivered to neurons within the brain.

8. The method of claim 1, wherein said AAV vector is administered intravascularly.

9. A method for treating a disease or disorder affecting the brain of a subject, said method comprising administering an adeno-associated virus (AAV) vector to said subject, wherein said AAV vector comprises a nucleic acid molecule encoding a therapeutic protein or inhibitory nucleic acid molecule and comprises hu.32 capsid protein or rh.8 capsid protein.

10. The method of claim 9, wherein said AAV vector comprises hu.32 capsid protein.

11. The method of claim 9, wherein said capsid protein comprises an amino acid sequence having at least 90% identity with SEQ ID NO: 1 or 3.

12. The method of claim 9, wherein said capsid protein comprises an amino acid sequence having at least 95% identity with SEQ ID NO: 1.

13. The method of claim 9, wherein said capsid protein comprises SEQ ID NO: 1.

14. The method of claim 9, wherein said nucleic acid molecule encodes a therapeutic protein.

16. The method of claim 9, wherein said disease or disorder is a lysosomal storage disease.

17. The method of claim 9, wherein said disease or disorder is a neurodegenerative disease.

18. The method of claim 9, wherein said nucleic acid molecule encodes a β-glucuronidase.

19. The method of claim 9, wherein said AAV vector is administered intravascularly.


SEQUENCE LISTING (4 sequences)

SEQ ID NO: 1 - protein, 736 aa, Dependovirus Adeno-associated virus
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 2211 bp, Dependovirus Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3 - protein, 736 aa, Dependovirus Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 4 - DNA, 2211 bp, Dependovirus Adeno-associated virus
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctattttggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggattacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcaatacacttccaactactacaaatctacaaatgtggactttgctgtcaacacggagggggtttatagcgagcctcgccccattggcacccgttacctcacccgcaacctgtaa